(19) United States
(12) Patent Application Publication
(10) Pub. No.: US 2025/0162968 A1
(43) Pub. Date: May 22, 2025

(54) Method for Producing Ionizable Lipids or Intermediates for the Synthesis of Such Lipids

(71) Applicant: NanoVation Therapeutics Inc., Vancouver (CA)

(72) Inventors: N. D. Prasad Atmuri, Vancouver (CA); Deaglan Arnold, London (CA); Fariba Saadati, Canada (CA); Daniel Kurek, Vancouver (CA); Jayesh Kulkarni, Vancouver (CA); Dominik Witzigmann, Binzen (DE); Marco A. Ciufolini, Vancouver (CA)

(73) Assignee: NanoVation Therapeutics Inc., Vancouver, BC (CA)

(21) Appl. No.: 18/834,843

(22) PCT Filed: Jan. 31, 2023
(86) PCT No.: PCT/CA2023/050129

§ 371 Date: Jul. 31, 2024

Related U.S. Application Data
(60) Provisional application No. 63/305,827, filed on Feb. 2, 2022.

(51) Int. Cl. C07C 45/57; B01J 31/38; C07C 51/09; C07C 51/38

(52) U.S. Cl. CPC C07C 45/57; B01J 31/38; C07C 51/09; C07C 51/38

(57) ABSTRACT

The present disclosure provides a method for producing one or more intermediates for the synthesis of one or more ionizable lipids, the method comprising: (i) producing a beta-ketoacid by reacting a cyclic ester, a terminal hydroxyester or a derivative thereof, a dicarboxylic acid half ester, or an acid chloride derivative of the dicarboxylic acid half ester, in a condensation reaction, thereby producing the beta-ketoacid or a beta-ketoester that is hydrolyzed to produce the beta-ketoacid; and (ii) decarboxylating the beta-ketoacid, thereby producing the one or more intermediates, wherein the one or more intermediates have a structure that may be defined by Formula A. Further provided is a method for producing ionizable lipid from the intermediate comprising adding an ionizable head group moiety to (a) a ketone group of one or more intermediates having the structure that may be defined by Formula A; or (b) a corresponding alcohol of the intermediate.

### CROSS-REFERENCE TO RELATED APPLICATIONS

This application claims priority from U.S. Ser. No. 63/305,827 filed on Feb. 2, 2022, which is incorporated herein by reference in its entirety.

### TECHNICAL FIELD

Provided herein is a method for producing ionizable lipids or intermediates for use in the production of such lipids.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment where the relevant translation machinery resides.

A key component of lipid nanoparticles is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the lipid nanoparticles are taken up by a cell by endocytosis, the ionizability of these lipids at low pH enables endosomal escape. This in turn allows the nucleic acid to be released into the intracellular compartment.

An early example of a lipid nanoparticle product approved for clinical use and reliant on ionizable lipid is Onpattro®, developed by Alnylam. Onpattro® is a lipid nanoparticle-based short interfering RNA (siRNA) drug for the treatment of polyneuropathies induced by hereditary transthyretin amyloidosis. Onpattro® is reliant on an ionizable lipid referred to as “DLin-MC3-DMA” or more commonly “MC3” by investigators (1, Scheme 1). This lipid has an ionizable dimethylamino head group, an ester linker and two C18 moieties derived from linoleic acid that converge into a single carbon atom.

The more recent Pfizer/BioNTech and Moderna covid-19 vaccines also rely on lipid nanoparticles to deliver mRNA to the cytoplasm of host cells. After entry into the host cell, the mRNA is transcribed to produce antigenic proteins. In the case of the covid19 vaccine, the mRNA encodes the Sars-Cov-2 spike protein. The ionizable lipid in the Pfizer/BioNTech, referred to as ALC-0315, 2, has a hydroxyl head group and a nitrogen atom that serves as anchoring point for branched lipid moieties. The structurally related ionizable lipid in the Moderna vaccine, referred to as SM-102, 3, exhibits reverse ester groups relative to ALC-0315.

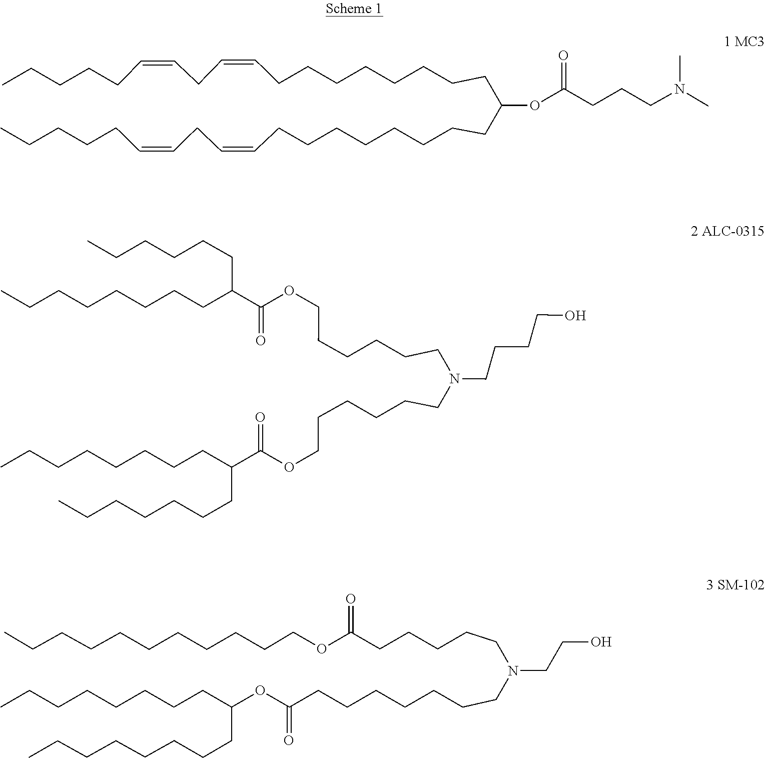

Other known ionizable lipids that are not components of approved drugs, but that remain valuable research tools in nucleic acid therapeutics, are depicted in Scheme 2 below. These include lipids 4 and 5 (Du, X. WO 2019/036000), as well as 6 (Harashima, H., et al., Acta Biomaterialia 2020, 102, 341).

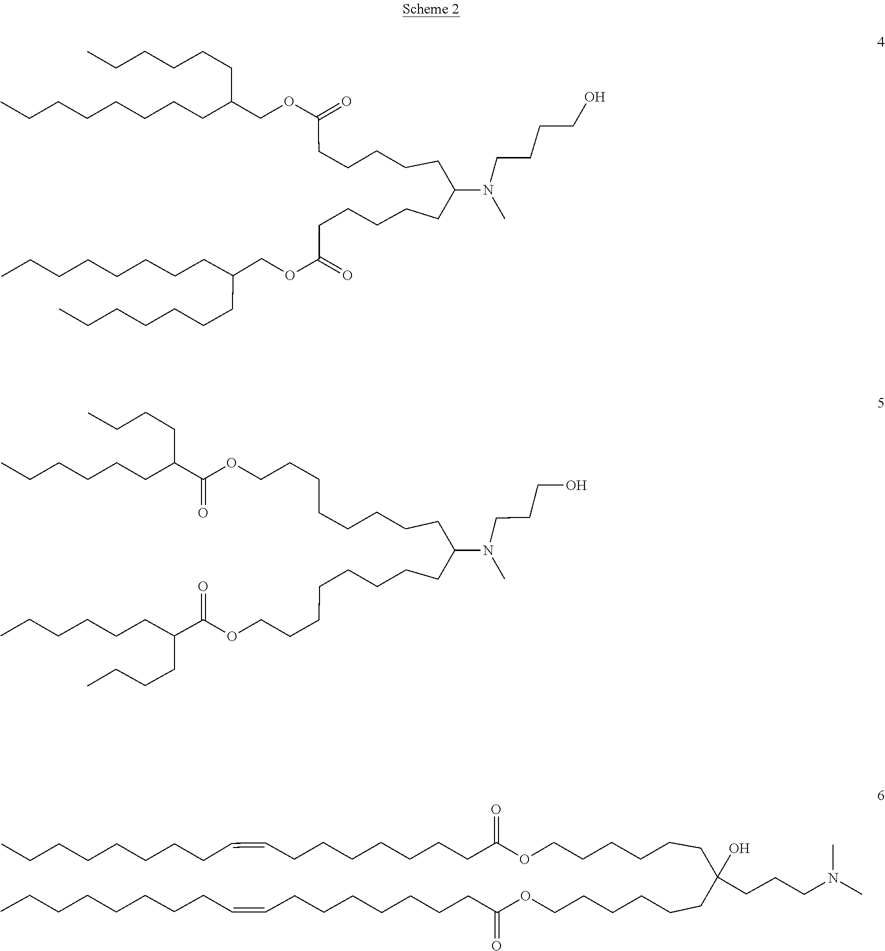 [SMILES: CCCCCCCCC(CCCCCC)COC(=O)CCCCCC(CCCCCC(=O)OCC(CCCCCCC)CCCCCCCC)N(C)CCCCO ; CCCCCCC(CCCC)C(=O)OCCCCCCCCC(CCCCCCCCOC(=O)C(CCCC)CCCCCC)N(C)CCCO ; CCCCCCCC/C=C\CCCCCCCC(=O)OCCCCCCC(O)(CCCCCCOC(=O)CCCCCCC/C=C\CCCCCCCC)CCCN(C)C]

While the foregoing ionizable lipids have proven efficacious, there remains an ongoing need to expand the repertoire of ionizable lipids available for the formulation of new therapeutic agents or prodrugs in a wider range of applications.

Further, limited attention has been given to developing efficient and cost-effective synthesis routes to make ionizable lipids. Ionizable lipids currently require multi-step reaction schemes that involve the use of hazardous chemicals, adding cost and complexity to their manufacture. For example, the synthesis of ALC-0315 includes the oxidation of an alcohol with pyridinium chlorochromate (PCC). PCC is a problematic chemical reagent based on hexavalent chromium, which is a known carcinogen. A more cost-effective and safer manufacturing method for ionizable lipids thus remains an unmet need in the industry.

The present disclosure seeks to address the shortcomings in the art and/or to provide useful alternatives to known methods for producing compounds, which can be further used to prepare a diverse range of ionizable lipids.

### Definitions

The following terms have the meanings ascribed to them unless specified otherwise.

As used herein, the term “lactone” refers to a cyclic ester possessing a chemical structure of the type shown as Formula I below, wherein:

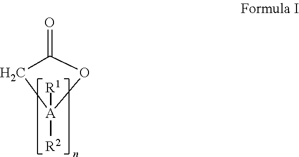

index n can range from 2 to 12;
A may be, independently:
   a carbon atom, in which case R¹ and R² are, independently, H or substituents such as alkyl, OH, O-alkyl, O—Si(alkyl)₃, S-alkyl, and N(alkyl)₂;
   a heteroatom such as a sulfur or oxygen atom, in which case R¹ and R² are absent;
   a C═C double bonds of E or Z geometry, in which case R¹ and R² are, independently, H or substituents such as alkyl, O-alkyl, S-alkyl.

As used herein, the term “hydroxyacyllactone” includes a compound of Formula II below, wherein the two groups in square brackets, i.e., [AR¹R²]ₙ, are identical, and wherein A, R¹, R², and index n, are as stated above for Formula I.

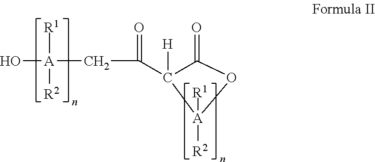

As used herein, the term “Claisen condensation of lactone” refers to the chemical process outlined in Scheme 3, whereby two molecules of a lactone of Formula I, in a suitable solvent and in the presence of an appropriate combination of reagents, merge through a chain of chemical events that are readily understood by the person skilled in the art ((a) Smith, M. B. March's Advanced Organic Chemistry, 7^{th} Ed.: John Wiley & Sons, Hoboken, NJ, 2013, p. 1234 ff. (b) Hauser, C. R., et al. Org React. 1942, 1, 266), leading to a product of Formula II:

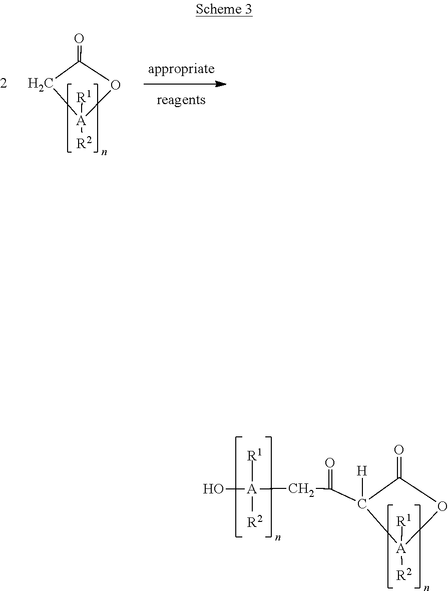

As used herein, the term “alkylation of the hydroxyacyllactone” and related expressions may refer to a chemical process whereby a hydroxyacyllactone of Formula II, in a suitable solvent and in the presence of an appropriate combination of reagents, is converted into a compound of Formula III, wherein R³ is a linear or branched alkyl group having from 1 to 20 carbon atoms, and optionally incorporating (i) from 0 to 4 heteroatoms, such as sulfur or oxygen atoms, (ii) from 0 to 3 C═C double bonds of E or Z geometry, and/or (iii) substituents such as OH, O-alkyl, O—Si(alkyl)₃, S-alkyl, and N(alkyl)₂ bonded to a carbon atom:

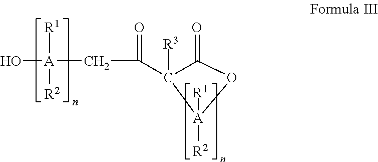

As used herein the term “terminal hydroxyester” includes a compound of Formula IV below, wherein A, R¹, R², and index n, are as stated above for Formula I, and R⁴ is a small alkyl containing from 1 to 5 carbon atoms, such as methyl, ethyl, propyl, isopropyl, butyl, isobutyl, tert-butyl, pentyl, and the like.

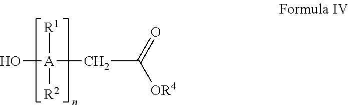

As used herein the term “dicarboxylic acid half ester” includes a compound of Formula V below, wherein A, R¹, R², and index n, are as stated above for Formula I, and R⁴ is a small alkyl containing from 1 to 5 carbon atoms, such as methyl, ethyl, propyl, isopropyl, butyl, isobutyl, tert-butyl, pentyl, and the like.

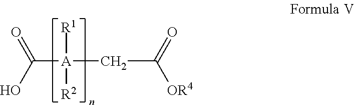

As used herein, the term “symmetrical bis-hydroxyalkyl ketone” includes a compound of Formula VI below, wherein the two groups in square brackets, i.e., [AR¹R²]ₙ, are identical, and wherein A, R¹, R², and index n, are as stated above for Formula I:

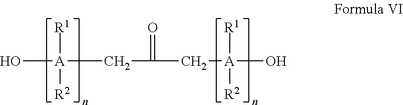

As used herein, the term “asymmetrical bis-hydroxyalkyl ketone” includes a compound of Formula VII below, wherein the two groups in square brackets, i.e., [AR¹R²]ₙ, are identical, wherein A, R¹, R², and index n, are as stated above for Formula I, and wherein R³ is a linear or branched alkyl group as stated above for Formula III:

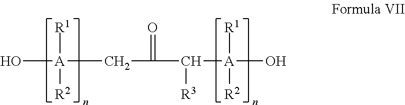

As used herein, the term “lipophilic acid” includes a compound of Formula VIII below, wherein R⁵ is a linear or branched alkyl group incorporating from 8 to 30 carbon atoms, and facultatively incorporating (i) from 0 to 4 heteroatoms, such as sulfur or oxygen atoms, (ii) from 0 to 3 C═C double bonds of E or Z geometry, and/or (iii) substituents such as OH, O-alkyl, O—Si(alkyl)₃, S-alkyl, and N(alkyl)₂ bonded to a carbon atom:

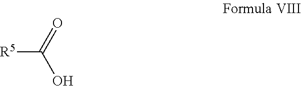

As used herein, the term “symmetrical ketodiester” derivative of a ketone of Formula VI includes a compound of Formula IX below, wherein the two groups in square brackets, i.e., [AR¹R²]ₙ, are identical, wherein A, R¹, R², and index n, are as stated above for Formula I, and wherein R⁵ is as defined above for the compound of Formula VIII.

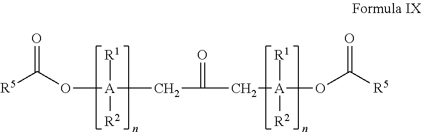

As used herein, the term “asymmetrical ketodiester” derivative of a ketone of Formula VII includes a compound of Formula X below, wherein the two groups in square brackets, i.e., [AR¹R²]ₙ, are identical, wherein A, R¹, R², R³ and index n, are as stated above for Formula III, and wherein R⁵ is as defined above for the compound of Formula VIII.

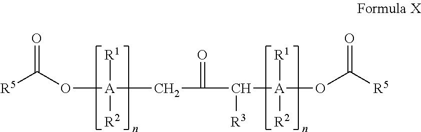

As used herein, the term “symmetrical ketodiacid” includes a compound of Formula XI below, wherein the two groups in square brackets, i.e., [AR¹R²]ₙ, are identical, wherein A, R¹, R², and index n, are as stated above for Formula I:

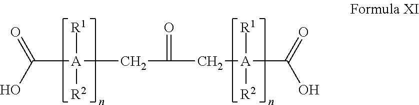

As used herein, the term “asymmetrical ketodiacid” includes a compound of Formula XII below, wherein the two groups in square brackets, i.e., [AR¹R²]ₙ, are identical, wherein A, R¹, R², and index n, are as stated above for Formula I, and wherein R³ is as stated above for Formula III.

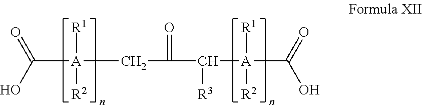

As used herein, the term “ketene dimer” includes a compound of Formula XIII below, wherein the two groups in square brackets, i.e., [AR¹R²]ₙ, are identical, wherein A, R¹, R², and index n, are as stated above for Formula I, wherein R⁴ is as stated above for Formula V, and wherein the wavy-line bond signifies that the double bond may be of E or Z geometry.

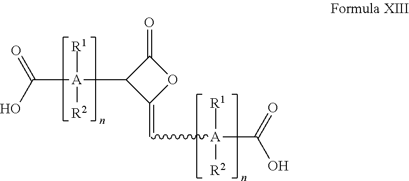

As used herein, the term “lipophilic alcohol” includes a compound of Formula XIV below, wherein R⁶ is a linear or branched alkyl group incorporating from 8 to 30 carbon atoms, and facultatively incorporating (i) from 0 to 4 heteroatoms, such as sulfur or oxygen atoms, (ii) from 0 to 3 C═C double bonds of E or Z geometry, and/or (iii) substituents such as OH, O-alkyl, O—Si(alkyl)₃, and N(alkyl)₂ bonded to a carbon atom:

R⁶—OH Formula XIV

As used herein, the term “symmetrical ketodiester” derivative of a keto-diacid of Formula XI includes a compound of Formula XV below, wherein the two groups in square brackets, i.e., [AR¹R²]ₙ, are identical, wherein A, R¹, R², and index n, are as stated above for Formula I, and wherein R⁶ is as defined above for the compound of Formula XIV.

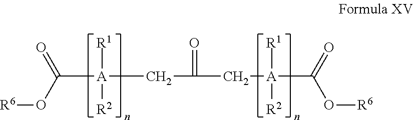

As used herein, the term “symmetrical ketodiester” derivative of a keto-diacid of Formula XII includes a compound of Formula XVI below, wherein the two groups in square brackets, i.e., [AR¹R²]ₙ, are identical, wherein A, R¹, R², and index n, are as stated above for Formula I, wherein R³ is as stated above for Formula III, and wherein R⁶ is as defined above for the compound of Formula XIV.

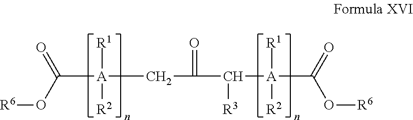

As used herein, the term “weak base” refers to a chemical species suitable for use in a given reaction step of the method described herein and which is capable of accepting a proton when placed in a solution, thereby producing a protonated form of itself, and such that the negative logarithm in base 10 of the aqueous ionization constant of said protonated form (i.e., its pKa) is between 4 and 13.

As used herein, the term “strong base” refers to a chemical species suitable for use in a given reaction step of the method described herein and which is capable of accepting a proton when placed in a solution, thereby producing a protonated form thereof, and such that the negative logarithm in base 10 of the aqueous ionization constant of said protonated form (i.e., its pKa) is greater than 13.

As used herein, the term “strong acid” refers to a chemical species suitable for use in a given reaction step of the method described herein and which is capable of donating a proton when placed in a solution, and such that the negative logarithm in base 10 of the aqueous ionization constant of said strong acid (i.e., its pKa) is lower than 3.

As used herein, the term “catalyst” refers to a chemical species that accelerates a reaction in a step of the method described herein, but that is not consumed in the course thereof. A catalyst thus allows the reaction to occur at a faster rate at lower temperatures.

As used herein, the term “ionizable lipid” refers to a lipid that, at a given pH, is in an electrostatically neutral form and that may either accept or donate protons, thereby becoming electrostatically charged, and for which the electrostatically neutral form has a calculated logarithm of the partition coefficient between water and 1-octanol (i.e., a cLogP) greater than 8.

As used herein, the term “ionizable head group moiety”, means a moiety that when incorporated within the ionizable lipid has at least one functional group that is capable of acquiring a net electrostatic charge, thereby becoming charged.

### SUMMARY

The present disclosure provides methods for the preparation of synthetic intermediates that serve as building blocks for the assembly of diverse ionizable lipids. Advantages of the methods outlined in the non-limiting examples set forth below include fewer chemical synthesis steps than conventional methods, steps that avoid or reduce the use of hazardous chemicals, and/or more economical routes to the desired lipids.

According to one aspect of the disclosure, there is provided a method for producing one or more intermediates for the synthesis of one or more ionizable lipids, the method comprising: (i) producing a beta-ketoacid by reacting a cyclic ester, a terminal hydroxyester or a derivative thereof, a dicarboxylic acid half ester, or an acid chloride derivative of the dicarboxylic acid half ester, in a condensation reaction, thereby producing the beta-ketoacid or a beta-ketoester that is hydrolyzed to produce the beta-ketoacid; and (ii) decarboxylating the beta-ketoacid, thereby producing the one or more intermediates.

In one embodiment, the one or more intermediates are a ketone. In another embodiment, the one or more intermediates have a structure as defined by Formula A hereinafter.

According to another aspect of the disclosure, there is provided a method for producing one or more ionizable lipids, the method comprising: (i) producing a beta-ketoacid by reacting a cyclic ester, a terminal hydroxyester or a derivative thereof, a dicarboxylic acid half ester, or an acid chloride derivative of the dicarboxylic acid half ester, in a condensation reaction, thereby producing the beta-ketoacid or a beta-ketoester that is hydrolyzed to produce the beta-ketoacid; (ii) decarboxylating the beta-ketoacid, thereby producing one or more ketone intermediates; and (iii) adding an ionizable head group moiety to (a) a ketone group of the one or more intermediates; or (b) a corresponding alcohol of the one or more intermediates, thereby producing the one or more ionizable lipids.

In one embodiment, the one or more intermediates are a ketone. In another embodiment, the one or more intermediates have a structure as defined by Formula A hereinafter.

In an embodiment of either aspect of the disclosure, the beta-ketoester is alkylated prior to being hydrolyzed to produce the beta-ketoacid.

In a further embodiment, the condensation reaction is a Claisen condensation of the cyclic ester, the terminal hydroxyester or the derivative thereof, or the dicarboxylic acid half ester to produce the beta-ketoacid or the beta-ketoester that is subsequently hydrolyzed to produce the beta-ketoacid.

In a further embodiment, the Claisen condensation is carried out in the presence of a catalyst and a weak base. For example, the catalyst may be AlCl₃, GaCl₃, TiCl₄, ZrCl₄, HfCl₄ or SnCl₄. In one embodiment, the catalyst is most advantageously TiCl₄. The weak base may be an amine, including tributylamine or triethylamine.

According to another embodiment, the condensation reaction comprises the conversion of the acid chloride to a ketene dimer by treatment with a weak base. The weak base may be an amine, including tributylamine, triethylamine or diisopropylethylamine.

Other objects, features, and advantages of the present disclosure will be apparent to those of skill in the art from the following detailed description.

### DETAILED DESCRIPTION

### Synthesis of Lipids Via Claisen Condensation

Symmetrical and asymmetrical bis-hydroxyalkyl ketones of Formula VI and Formula VII are valuable building blocks for the preparation of ionizable lipids, examples of which are described in more detail herein. The method of embodiments of the present disclosure rests at least in part on the observation that lactones, such as those of Formula I, undergo Claisen condensation to produce a hydroxyacyllactone of Formula II.

The Claisen condensation of lactones described in non-limiting examples of the present disclosure is exemplified in Scheme 4 with caprolactone, 7. In this non-limiting embodiment, the condensation reaction affords 8.

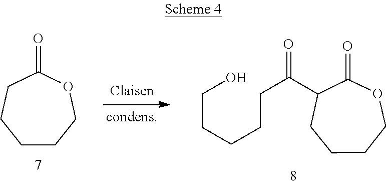 [SMILES: O=C1CCCCCO1 ; O=C(CCCCCO)C1CCCCOC1=O]

Compounds such as 8 can optionally be subjected to various transformations that produce valuable building blocks for the synthesis of various ionizable lipids. One such optional transformation is silyl protection of the OH group and alkylation of the resulting 9. Without intending to be limiting,

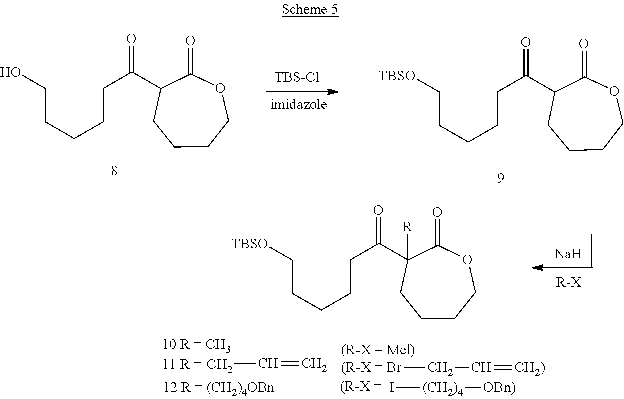

this is exemplified in Scheme 5 with the conversion of 8 into tert-butyldimethylsilyl (TBS) derivative 9 and alkylation thereof to 10-12. Furthermore, alkylated derivatives such as 10-12 are amenable to selective release of the silyl group or to selective opening of the lactone and consequent decarboxylation. Without intending to be limiting, this is exemplified in Scheme 6 with the conversion of 10 into either 13 or 14.

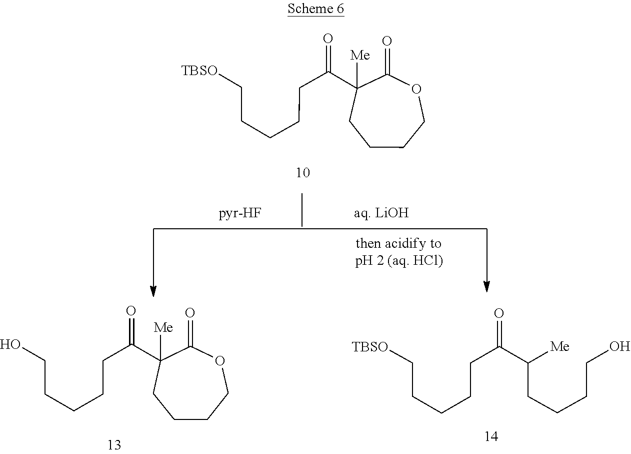

Another optional transformation of Claisen products such as 8 is conversion of the OH group into a good nucleofuge and displacement thereof with a nucleophile. Without intending to be limiting, this is exemplified in Scheme 7 with the conversion of 8 into tosylate 15 or mesylate 16, followed by displacement with thioacetate ion. Product 17 thus obtained is a valuable building block for the synthesis of certain ionizable lipids described in co-pending and co-owned Provisional Patent Application No. 63/434,506 filed on Dec. 22, 2022, incorporated herein by reference.

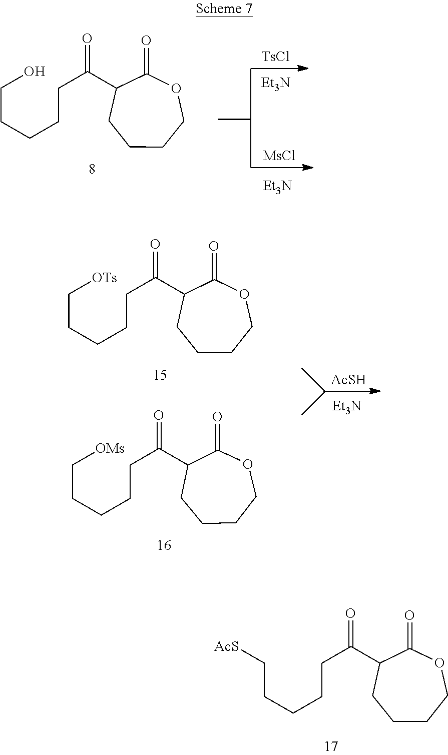

In certain embodiments, it is expedient not to isolate the immediate product of the Claisen condensation, but to subject it to lactone hydrolysis and decarboxylation in situ, resulting in direct formation of a symmetrical bis-hydroxyalkyl ketone. Without intending to be limiting, this is exemplified in Scheme 8 with the conversion of 8 into bis-hydroxyalkyl ketone 19. Thus, addition of water to the solution in which the Claisen reaction took place, removal of the organic solvent (rotary evaporation), and heating of the aqueous residue containing 8, induces lactone opening and decarboxylation of the intermediate ketoacid 18 to produce 19 directly.

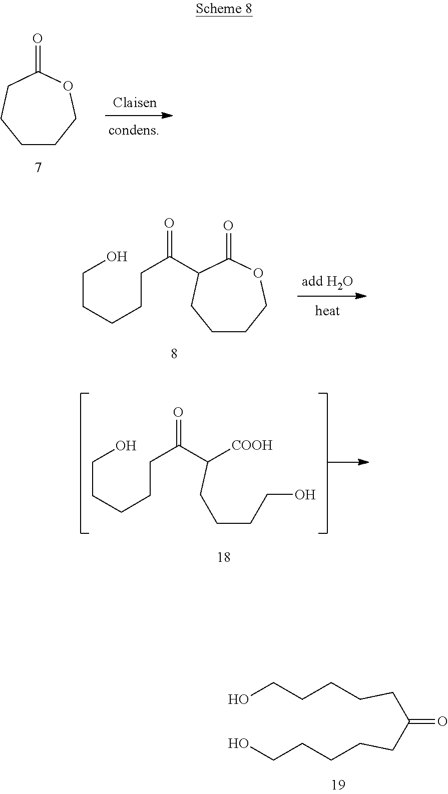

A number of lactones of Formula I are commercially available. Those that are not readily available can be readily prepared by those of skill in the art via a Baeyer-Villiger oxidation of suitable cyclic ketones. Without intending to be limiting, this is exemplified in Scheme 9 by the conversion of cycloheptanone, 20, into lactone 21 upon reaction with a peracid such as MCPBA (Duan, J., et al.; Chem. Sci. 2019, 10, 8706), and of cyclooctanone, 22, into lactone 23 upon reaction with a peracid such as peracetic acid.

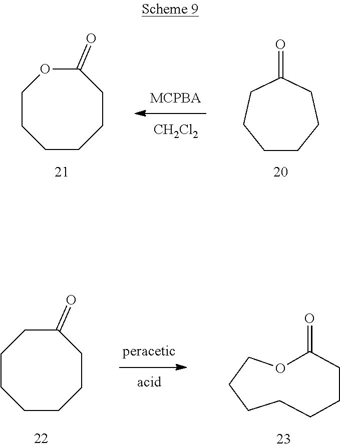

There may be instances when neither a lactone nor a suitable cyclic ketone is readily available, or the cyclic ketone is unsuitable for the conduct of a Baeyer-Villiger oxidation. In such embodiments, the inventors have found that the desired dihydroxyketones can still be manufactured starting with a Claisen condensation of a derivative of a terminal hydroxyester (e.g.,

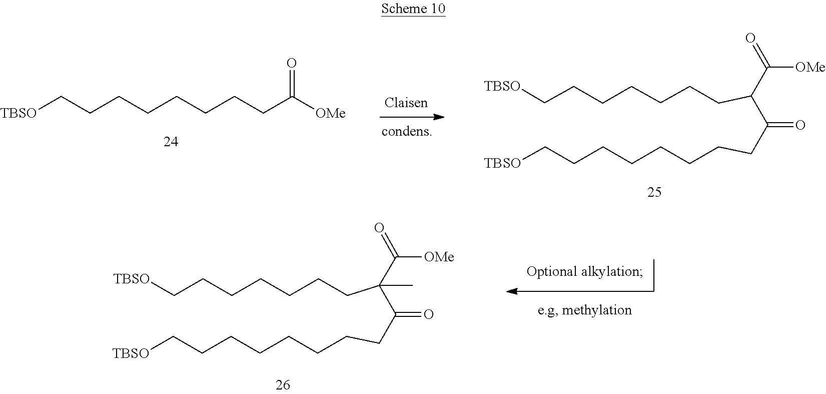 [SMILES: COC(=O)CCCCCCCCO[Si](C)(C)C(C)(C)C ; COC(=O)C(CCCCCCCO[Si](C)(C)C(C)(C)C)C(=O)CCCCCCCCO[Si](C)(C)C(C)(C)C ; COC(=O)C(C)(CCCCCCCO[Si](C)(C)C(C)(C)C)C(=O)CCCCCCCCO[Si](C)(C)C(C)(C)C]

Formula IV), in which the OH group is protected as a trialkylsilyl ether, such as a tert-butyldimethylsilyl ether, or an alkyl ether such as a benzyl ether. Without intending to be limiting, this is exemplified in Scheme 10 with the tert-butyldimethylsilyl (TBS) ether of methyl 4-hydroxynonanoate, 24. The Claisen condensation leads to compound 25, which may be optionally alkylated at the C atom between the two carbonyl groups to produce 26. Substances 25 or 26 can then be converted into bis-hydroxyalkyl ketones 29-30 as shown in Scheme 11 below. The first step, hydrolysis of the ester leading to ketoacids 27, may be carried out under conditions that also induce release of the tert-butyldimethylsilyl protecting groups. Accordingly, structure 27 may comprise a mixture of fully protected (both R¹ groups are trialkylsilyl), partially deprotected (only one of the two R¹ groups is a trialkylsilyl, the other is H), and fully deprotected (both R¹ groups are H) species. Complete deprotection may be achieved in a subsequent, separate step. Acids 27 easily undergo decarboxylation upon heating, producing ketones of the type 28. Any surviving trialkylsilyl ether may then be released by any of the methods well known to the person skilled in

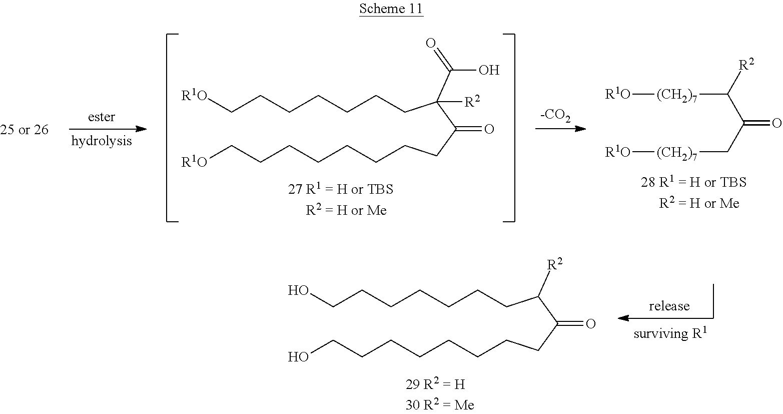

the art to produce ketones 29-30. No release of the O-protecting groups occurs when the sequence is carried out with substrates of the type 24 in which an alkyl ether, such as a benzyl ether, is present in lieu of a trialkylsilyl ether. In such cases, products of the type 27 and 28 retain both O-protecting groups (e.g., R¹ is a benzyl group), and a final step is required to release said protecting groups and produce 29-30.

In further aspects of the disclosure, an ionizable lipid is prepared from symmetrical or asymmetrical ketodiacids, such as those of Formula XI and XII. These compounds have been prepared by routes that involve the dialkylation of a 3-ketoglutarate ester, followed by hydrolysis and decarboxylation (Ansell, S. M., et al., WO 2013/086322 A1; incorporated herein by reference), or Claisen condensation of, e.g., N,N-dimethylamide derivatives of the free carboxylic acid terminus promoted by an alkoxide base at elevated temperature, followed by vigorous acid treatment (Cohen, H., et al., J. Org. Chem. 1973, 38, 1424; incorporated herein by reference).

According to certain embodiments of the present disclosure, ketodiacids of Formula XI and Formula XII can be advantageously synthesized via a Claisen condensation of diacid half esters of Formula V. Several diacid half esters of Formula V are commercially available compounds. Those that are not readily available can be made by mono-saponification of corresponding diesters, which are more readily available, with a metal hydroxide. Without intending to be limiting, this is exemplified in Scheme 12 by the preparation of monomethyl azelate, 32, from dimethyl azelate, 31, as described by Vozdvizhenskaya (Vozdvizhenskaya, O. A.; et al. Chem. Het. Comp. 2021, 57, 490; incorporated herein by reference).

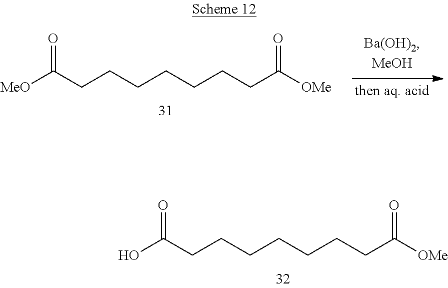 [SMILES: COC(=O)CCCCCCCC(=O)OC ; COC(=O)CCCCCCCC(=O)O]

Without intending to be limiting, the Claisen condensation of a dicarboxylic acid half ester is exemplified in Scheme 13 by the conversion of monomethyl sebacate, 33, into compounds 37-38.

According to other embodiments of the present disclosure, ketodiacids of Formula XI and

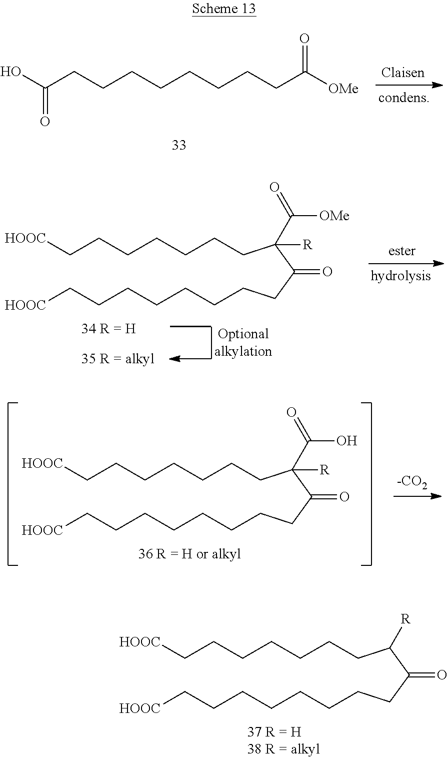

Formula XII can be synthesized even more advantageously from a ketene dimer obtained by dehydrohalogenation of a half-ester/half acid chloride derivative of a dicarboxylic acid monoester (Sauer, J. C. J. Am. Chem. Soc. 1947, 69, 2444; incorporated herein by reference). Without intending to be limiting, this is exemplified in Scheme 14 with the synthesis of compounds 42 and 45 from the acid chloride derivative 39 of monomethyl sebacate, 33, and the corresponding ketene

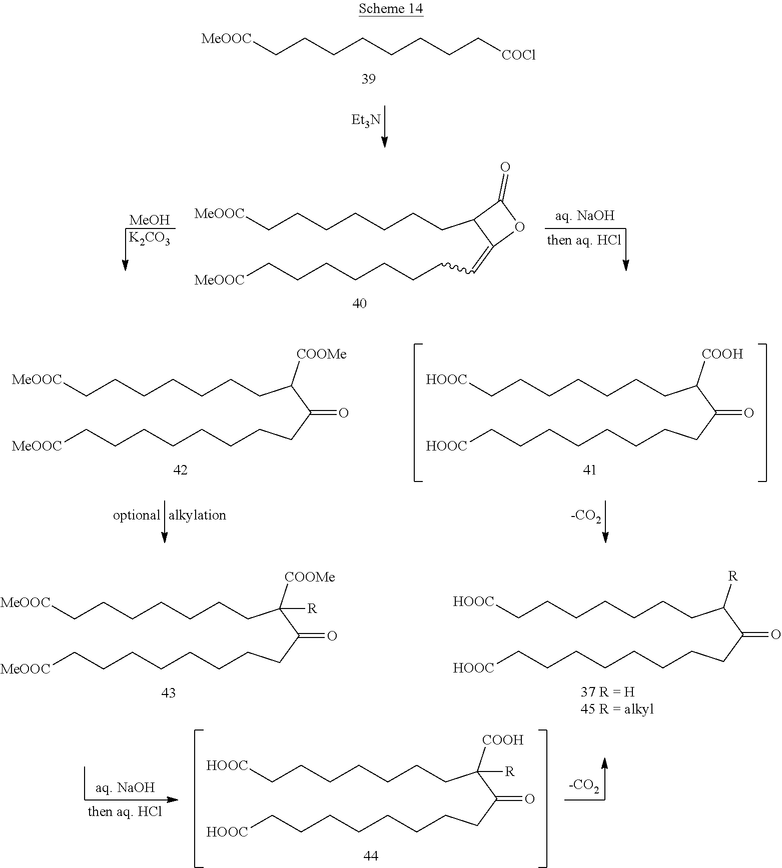

dimer, 40. The wavy bond in the structure of the latter signifies that the double bond can be of E- or Z-configuration.

The advantages of embodiments of the methods in the present disclosure are outlined in Examples 1-6 below. Such examples set forth comparative synthesis schemes for the preparation of building blocks that may be further subjected to organic synthesis to produce cationic and anionic ionizable lipids. The production of representative bis-hydroxyalkyl ketones and ketodiacids are given for the purpose of illustration only and not by way of limitation on the scope of the invention.

The examples herein demonstrate the ease in which the foregoing compounds can be synthesized. Examples 1, 2 and 5 are comparative examples that illustrate the improved economics of the methods disclosed herein relative to known organic syntheses to prepare the foregoing ketones.

General considerations. The Claisen condensation is commonly carried out in the presence of strong bases (e.g., alkoxides or e.g., sodium hydride, NaH) at elevated temperatures (120-150 degrees C.). While the use of NaH is a particularly effective reagent for traditional Claisen condensation, it poses a number of safety hazards, especially when the reaction is operated at elevated temperature, and thus is best avoided in certain embodiments. In some embodiments, the present disclosure provides a synthetic route based on a variant of the Claisen condensation that occurs under mild conditions. Such a method involves the use of weakly basic agents (e.g., amines) at or near room temperature; e.g., from −10 to +40° C., thus circumventing the safety hazards posed by operation with NaH at high temperature.

### EXAMPLES

Provided below is Formula A representing intermediates for lipid synthesis that are more readily and more economically available via the synthetic technology described herein:

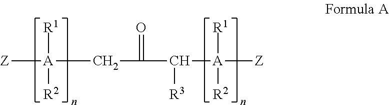

wherein:
integer index n ranges from 2 to 12;
A is, independently:
   a carbon atom, in which case R¹ and R² are, independently, H, or alkyl, OH, O—Si(alkyl)₃, O-alkyl, S-alkyl, or N(alkyl)₂;
   a sulfur or oxygen atom, in which case R¹ and R² are absent;
   a C═C double bonds of E or Z geometry, in which case R¹ and R² are, independently, H or alkyl, O-alkyl, O—Si(alkyl)₃, S-alkyl,
R³ is H or a linear or branched alkyl group having from 1 to 20 carbon atoms, optionally incorporating (i) from 0 to 4 sulfur or oxygen atoms, (ii) from 0 to 3 C═C double bonds of E or Z geometry, (iii) OH, O-alkyl, O—Si(alkyl)₃, S-alkyl, N(alkyl)₂ substituents bonded to a carbon atom, and/or (iv) a linear or branched alkyl substituent having less than 6 carbon atoms; and
Z is OH or COOH.

### Experimental Protocols

Examples 7-20 below provide representative experimental procedures for the preparation of intermediates of Formula A. Unless otherwise specified, all reagents and solvents were commercial products and were used without further purification, except THF (freshly distilled from Na/benzophenone under Ar), CH₂Cl₂ (freshly distilled from CaH₂ under Ar). “Dry methanol” was freshly distilled from magnesium turnings. All reactions were performed under a nitrogen or argon atmosphere. Reaction mixtures from aqueous workups were dried by passing over a plug of anhydrous Na₂SO₄ held in a filter tube and concentrated under reduced pressure on a rotary evaporator. Thin-layer chromatography was performed on silica gel plates coated with silica gel (Merck 60 F254 plates) and column chromatography was performed on 230-400 mesh silica gel. Visualization of the developed chromatogram was performed by staining with I₂ or potassium permanganate solution. ¹H and ¹³C nuclear magnetic resonance (NMR) spectra were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the δ scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionisation (FD/FI) mode.

### Example 1: Comparative Example of Known Syntheses of 1,11-dihydroxy-6-undecanone, 19, Vs the Inventive Synthetic Scheme

Compound 19 has been prepared (Momenteau, M., et al. J. Chem. Soc., Perkin Trans. I 1988, 283) from diacid 51 (Scheme 15), which can be made in four steps from dimethyl adipate,

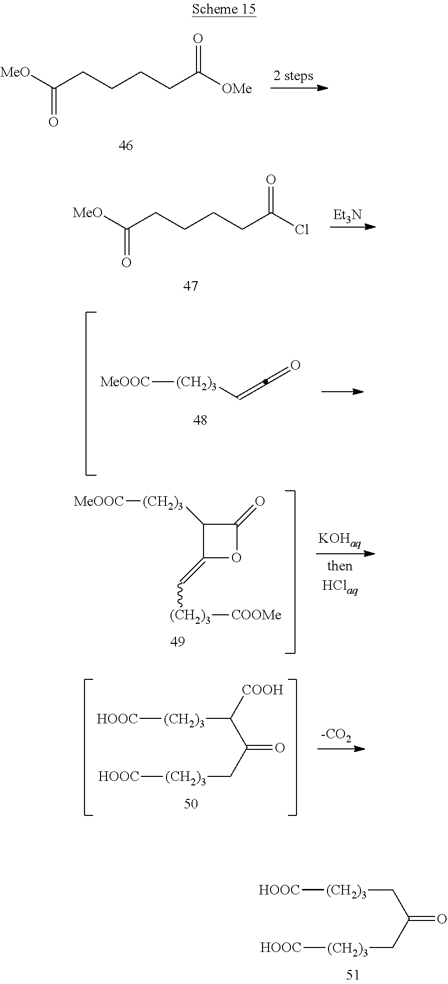

46, as described in an Organic Synthesis procedure (Durham, L. J., et al., Org. Synth., Coll. Vol. 4, 1963, 555; incorporated herein by reference). Compound 51 is then esterified and ketalized to produce 52 (Scheme 16), which is reduced with lithium aluminum hydride (LAH). The ketal in product 57 thus obtained is hydrolyzed to produce 19, which is thus available in a total of 7 steps from dimethyl adipate.

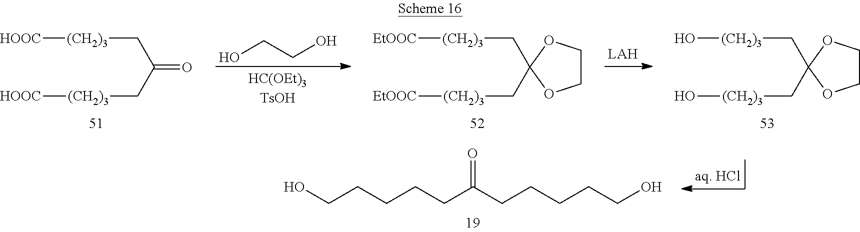

A more recent synthesis reported by Oniciu (US 2021/0024447) is shorter (3 steps) but requires costly reagents. Thus, protection of 5-bromo-1-pentanol, 54, ($3-5/g) as THP ether 55 sets the stage for a double alkylation of tosylmethylisocyanide in the presence of moisture-sensitive sodium tert-amyloxide (˜$1/g). Treatment of the resultant 56 with aqueous HCl causes release of the THP groups and of the TosMIC-derived fragments to provide the desired compound 19 (Scheme 17).

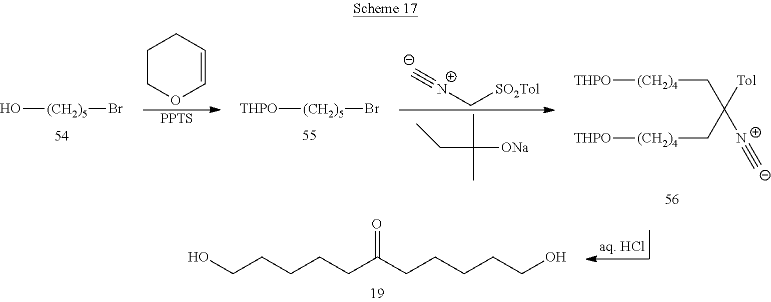

Routes involving alkene metathesis (Kranidiotis, N. S.; et al., Heterocycles 2018, 96, 1441) are longer and economically unattractive.

In contrast to the foregoing, certain embodiments of the method of the present disclosure allow the preparation of compound 19 in one step from economical caprolactone, 7 (Scheme 18). Thus, Claisen condensation of 7 provides 8, which is capable of being isolated. However, it is expedient to transform 8 to 19 by adding water to the reaction solution and heating the resultant mixture.

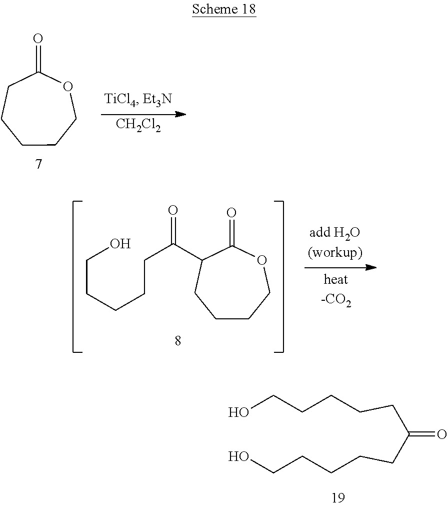

This causes hydrolysis of TiCl₄ and formation of an aqueous solution of HCl, which induces hydrolysis of the lactone ring in 8. Applied heat results in decarboxylation of the nascent beta-ketoacid, leading directly to 19.

In a like manner, lactones 21 and 23 of Scheme 9 can be converted into hydroxyacyl lactones 57 and 59, which may be isolated as described earlier for 9 or transformed directly into bis-dihydroxyketones 58 and 60 (Scheme 19)

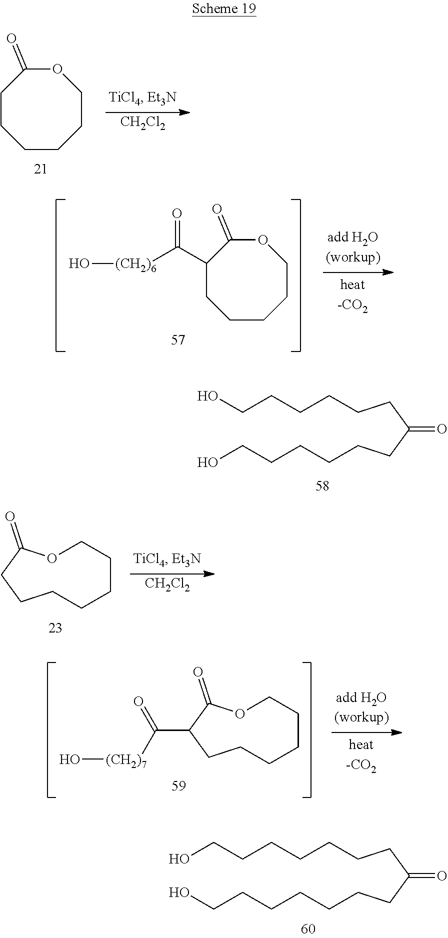

### Example 2: Comparative Example of Known Syntheses of 1,19-dihydroxynonadecan-10-one, 67, Vs the Inventive Synthetic Scheme

As described by, e.g., Ansell, et al (WO 2017/117528 A1), 1,19-dihydroxynonadecan-10-one, 67 can be made starting with protection of 9-bromo-1-nonanol, 61, as THP ether 62 (Scheme 20).

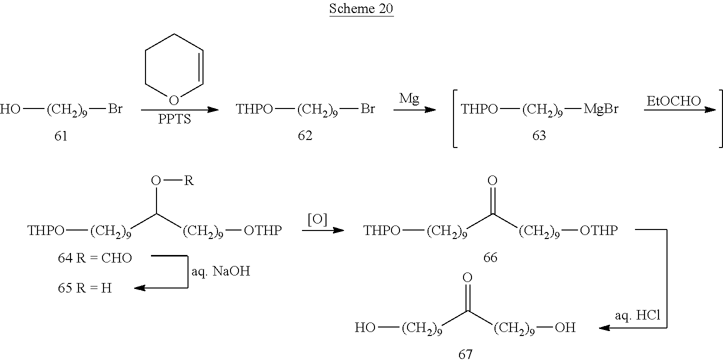

Compound 62 is reacted with metallic magnesium to produce Grignard reagent 63. While Grignard reagents are commonly used in the pharmaceutical industry, their nature and properties impose a series of chemical and safety precautions that translate into additional production costs. Consequently, they are best avoided in pharmaceutical manufacturing practice. Grignard reagent 63 is then caused to react in situ with ethyl formate, leading to a mixture of desired alcohol 64 and the corresponding formate ester 65. The mixture of 64 and 65 is treated in a separate step with aqueous NaOH. This releases the formyl group from 64 and converts it into alcohol 65. Oxidation of the alcohol produces 66. Oxidation steps are best avoided in pharmaceutical manufacturing practice. Compound 66 is then deprotected with aqueous HCl to afford the ultimate 67. The route to 63 thus involves a total of only 4 synthesis steps, but suffers from the disadvantage of requiring a Grignard reaction and an oxidation step.

The method of embodiments of the present disclosure bypass the Grignard reaction and the oxidation step. A further advantage of embodiments of the inventive method is that a starting ester of Formula IV may be utilized when neither a lactone nor a corresponding cyclic ketone is readily available. To illustrate, compound 67 could be obtained by Claisen condensation of lactone 69 (Scheme 21). This lactone is not commercially available, but it could be prepared by Baeyer-Villiger oxidation of cyclodecanone, 68. Unfortunately, the latter compound is exceedingly costly (>$50/g). A route to 67 from 68-69 would thus be uneconomical.

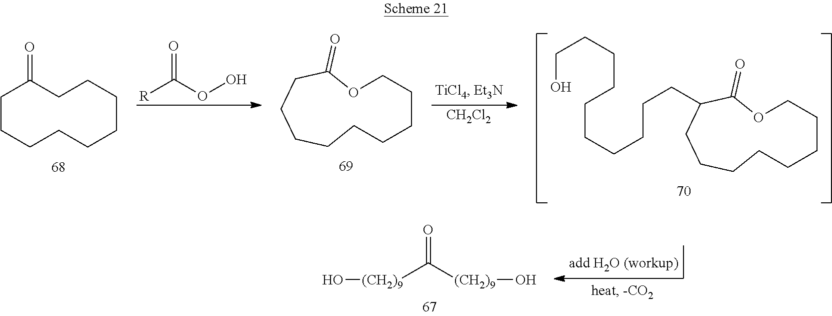

In contrast, inexpensive dimethyl sebacate, 71 ($0.06/g), can be converted into the monoester, 33 (Vozdvizhenskaya, O. A.; et al., Chem. Het. Comp. 2021, 57, 490). Subsequent reaction of 33 with borane, either as BH₃·SMe₂ or BH₃·THF, results in selective reduction of the carboxylic acid to

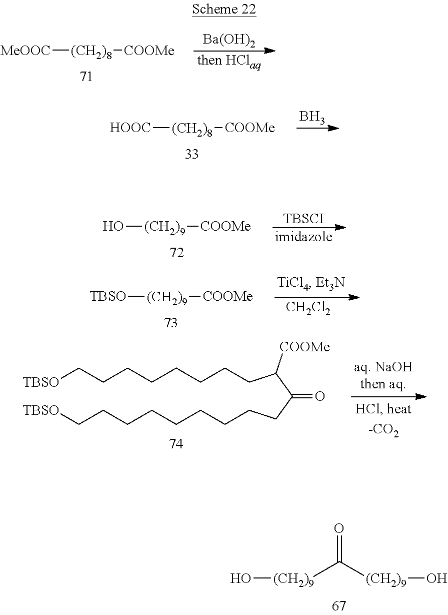

alcohol 72. Protection of the OH group in 72 as a tert-butyldimethylsilyl ether and Claisen condensation of the resultant 73 produces 74. Hydrolysis of the ester occurs concomitantly with release of the TBS groups, and final decarboxylation leads directly to 67 in 5 steps from 71 (Scheme 22).

### Example 3: Synthesis of 1,11-dihydroxy-5-methylundecan-6-one, 75, by the Inventive Synthetic Scheme

The preparation of an asymmetrical bis-hydroxyalkyl ketone such as 1,11-dihydroxy-5-methylundecan-6-one, 75, by the method of the present disclosure entails heating compound 10 of Scheme 6 with an aqueous solution of a strong mineral acid, for example, 3 N aqueous HCl, resulting in release of the TBS group, hydrolysis of the lactone, and decarboxylation of the intermediate ketoacid to produce 75 (Scheme 23).

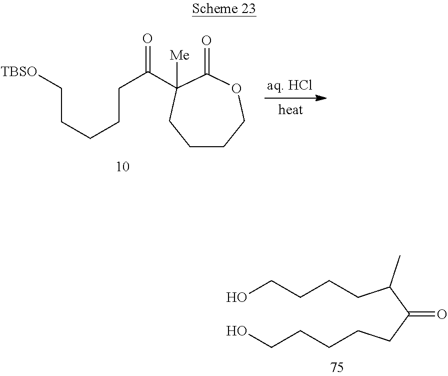 [SMILES: CC1(C(=O)CCCCCO[Si](C)(C)C(C)(C)C)CCCCOC1=O ; CC(CCCCO)C(=O)CCCCCO]

### Example 4: Synthesis of 1,19-dihydroxy-9-methylnonadecan-10-one, 77, by the Inventive Synthetic Scheme

The preparation of an asymmetrical bis-hydroxyalkyl ketone such as 1,19-dihydroxy-9-methylnonadecan-10-one, 77 by the method of the present disclosure entails the methylation of the enolate of 74 to produce 76, which can be isolated (Scheme 24). However, it is expedient to convert 76 into 77 in situ by ester hydrolysis, release of the TBS groups, and decarboxylation of the intermediate beta-ketoacid.

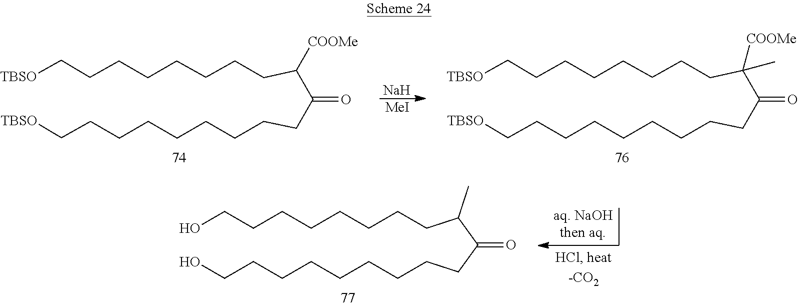

### Example 5: Comparative Example of Known Syntheses of 10-oxo-nonadecanoic Acid, 37, Vs the Inventive Synthetic Scheme

The preparation of ketodiacid 37 shown earlier in Scheme 14 has been described (see (a) Ansell, S. M., et al., WO 2013/086322 A1; (b) Tanabe, S., et al., WO2019/235635 A1; and (c) Endo, T., et al., WO 2020/246581 A1). The procedures utilize the method outlined in Scheme 25. Thus, double alkylation of diethyl 3-ketoglutarate, 78, with ethyl 8-bromooctanoate, 79 (>$3/g) in the presence of air- and moisture-sensitive NaOEt leads to 80, which upon acidic hydrolysis and decarboxylation produces 37.

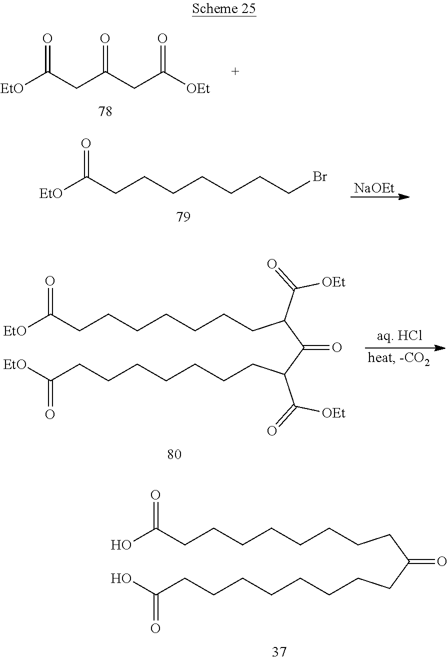

In contrast, economical monomethyl sebacate, 33, available as shown earlier in Scheme 16, undergoes Claisen condensation to produce 34, as shown earlier in Scheme 13. Subsequent hydrolysis and decarboxylation of 34 leads to 37 (Scheme 26).

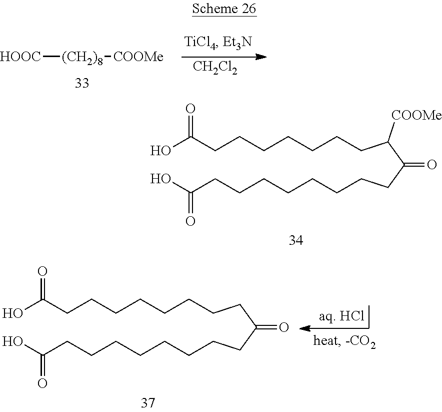

Diacid half esters such as 33 that do not contain other reactive functional groups, such as free alcohols and/or amines, can be advanced to ketodiacids of the type 37 even more advantageously by a method described in Organic Syntheses (Durham, L. J., et al., Org. Synth., Coll. Vol. 4, 1963, 555), as shown earlier in Scheme 14. This is illustrated in Scheme 27 by the conversion of 33 into 37 by said method. Thus, the COOH group in 33 is converted into the corresponding acid chloride 39, which upon treatment with triethylamine in toluene forms ketene 81. The latter dimerizes in situ to produce 40, which can be isolated, and wherein the wavy bond signifies that the C═C double bond may be of E or Z configuration. Hydrolysis of the beta-lactone in 40 and decarboxylation of the resulting ketoacid produces 37.

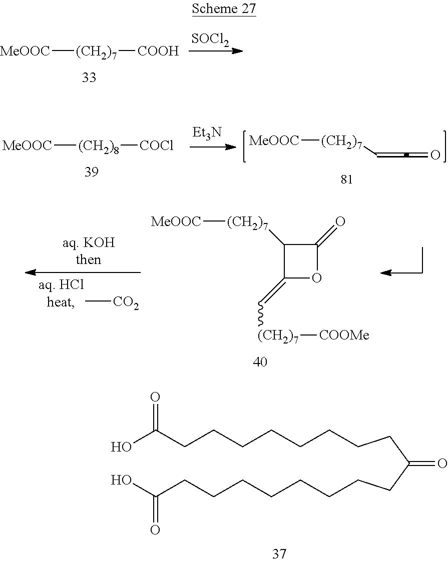

### Example 6: Syntheses of 9-methyl-10-oxo-nonadecanoic Acid, 83, by the Inventive Synthetic Scheme

The preparation of 83 by embodiments of the methods of the present disclosure (Scheme 28) can be achieved starting with the conversion of ketene dimer 40 into beta-ketoester 42, followed by methylation of the enolate of 42 to give 82 (Scheme 22). While 82 can be isolated, it is expedient to convert it directly to 83 by ester hydrolysis and decarboxylation.

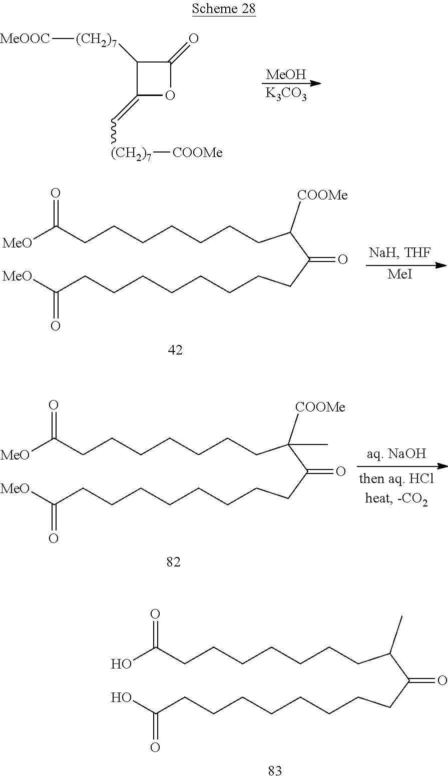

The compounds produced in Examples 1-6 can be used to prepare ionizable lipids through methods that are well understood by those skilled in the art. The schemes below serve to illustrate, without intending to be limiting, representative types of ionizable lipids that may thus be prepared.

Bis-hydroxyalkyl ketones of Formula VI or VII can be used to synthesize ionizable lipids starting with the esterification of the OH groups with a lipophilic acid, such as described in Formula VIII (R⁵—COOH), either in the presence of an appropriate coupling agent, such as a carbodiimide, or by the use of acyl chloride or mixed anhydride derivatives of R⁵—COOH. Furthermore, the ketone group in the ketodiesters thus produced can be optionally reduced in selective reactions, e.g., with NaBH₄ in an appropriate solvent, to provide the corresponding alcohol. A suitable ionizable head group moiety may subsequently be attached to either the ketone or the alcohol to produce an ionizable lipid. Without intending to be limiting, the process is exemplified in Scheme 29 with ketones 19 and 75.

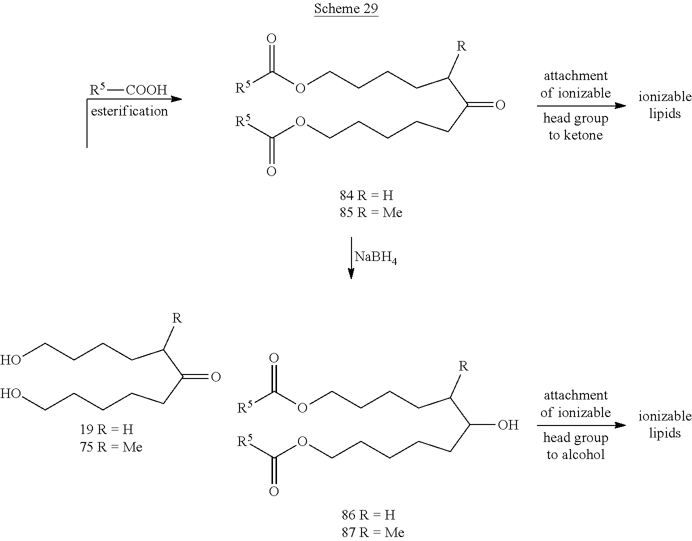

An ionizable head group that may be attached to the representative ketones 84-85 of Scheme 29 above is exemplified in Scheme 30 by the preparation of ionizable lipids 92-93. The displacement of sulfonate groups from compounds of the type 90-91 may be optionally carried out under microwave (mw) irradiation as described by Buschmann (Buschmann, M. D., et al., Commun. Biol. 2021, 4, 956; https://doi.org/10.1038/s42003-021-02441-2; incorporated herein by reference). Actual examples of the transformations outlined in Scheme 30 may be found in co-pending and co-owned U.S. Provisional Patent Application No. 63/410,273 filed on Sep. 27, 2022, incorporated herein by reference.

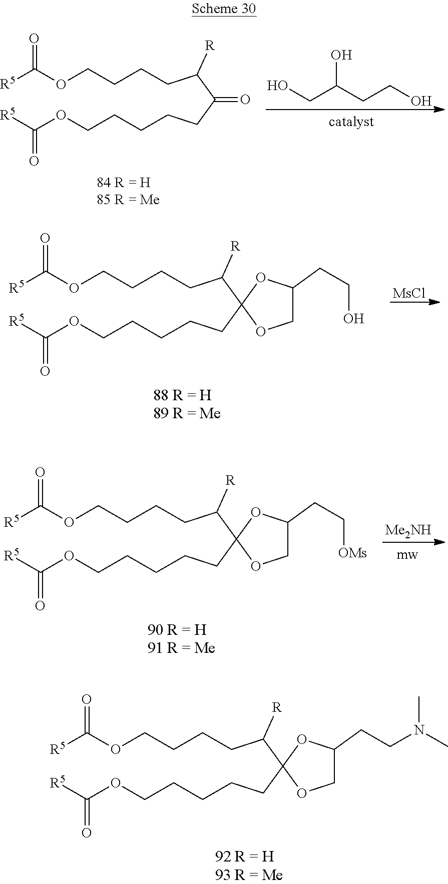

Without intending to be limiting, Scheme 31 exemplifies another kind of ionizable head group that may be introduced from representative ketones 84-85, through reductive amination with an aminoalcohol such as 94, wherein R′ is either H or a small alkyl and Z is either H or a protecting

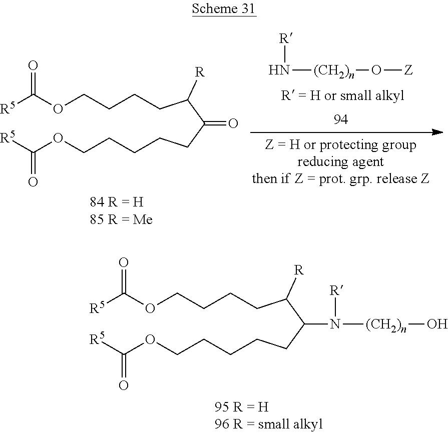

group. If Z is H, then products 95-96 are formed directly in the foregoing reaction. If Z is a protecting group, the products of the foregoing reaction are subjected to a treatment that releases Z, thus producing 95-96. Actual examples of the transformations outlined in Scheme 31 may be found in co-pending and co-owned U.S. Provisional Patent Application No. 63/410,273 filed on Sep. 27, 2022, incorporated herein by reference.

A number of aminoalcohols of the type 94 wherein R′ is a small alkyl are costly. In such cases, it may be most advantageous to perform the reductive amination of ketones 84-85 with an amine of the type 94 wherein R′ is H (Scheme 32), then N-alkylate compounds 97-98 thus obtained, for example via a second reductive alkylation step. Actual examples of the transformations outlined in Scheme 32 may be found in co-pending and co-owned patent application U.S. Provisional Patent Application No. 63/410,273 filed on Sep. 27, 2022, incorporated herein by reference.

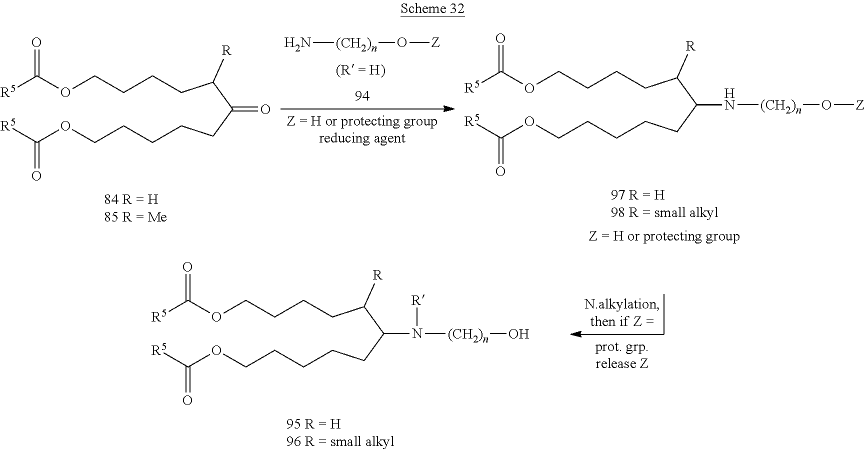

A non-limiting example of an ionizable head group that may be attached to alcohols 86-87 shown earlier in Scheme 29 is a dialkylaminoacyl group provided by a carboxylic acid such as 99 or a corresponding mixed anhydride, or a corresponding acid chloride (Scheme 33). This is exemplified by the conversion of 86-87 into ionizable lipids 100-101, either by reaction with acid 99 in the presence of a condensing agent, or by reaction with a mixed anhydride of 99, or by reaction with the acid chloride of 99, in all cases in the presence of a weak base. Actual examples of the transformations outlined in Scheme 33 may be found in co-pending and co-owned U.S. Provisional Patent Application No. 63/410,273 filed on Sep. 27, 2022, incorporated herein by reference,

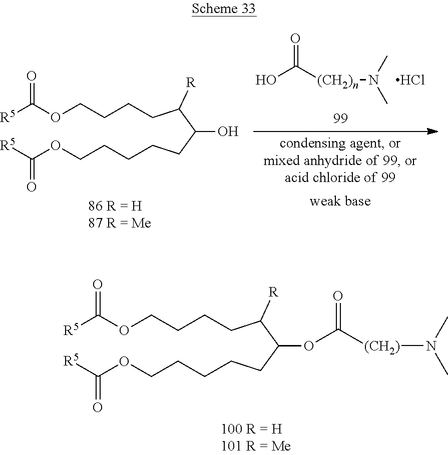

In the foregoing examples, the acyl groups R⁵—CO are identical. However, symmetrical bis-hydroxyalkyl ketones such as 19 can be readily mono-esterified as described in a co-owned and co-pending U.S. Provisional Patent Application No. 63/410,273 filed on Sep. 27, 2022, incorporated herein by reference. Thus, monoesterification with a generic lipophilic acid R⁵—COOH converts 19 into 102 (Scheme 34).

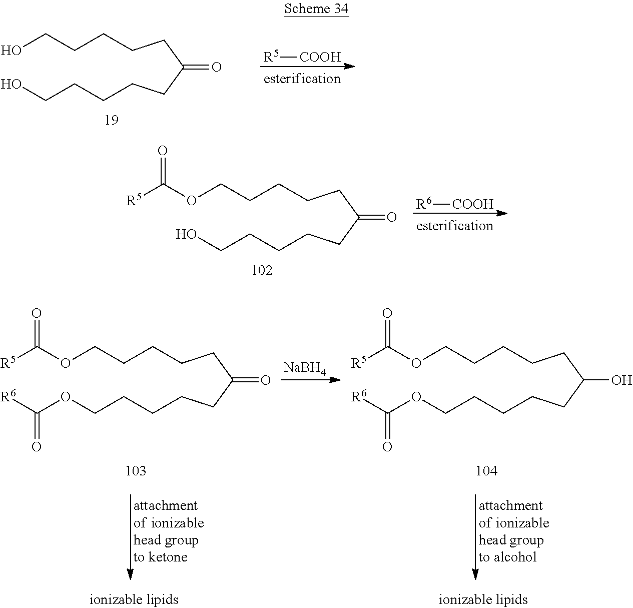

The OH group in compound 102 can then be esterified with a second lipophilic acid, R⁶—COOH, resulting in formation of diester 103, wherein the two acyl groups are different. The ketone in 103 can be further reduced to alcohol 104. Ketone 103 and alcohol 104 can then be transformed into ionizable lipids by the methods shown in Schemes 30-33 above.

Those skilled in the art will appreciate that the mono-esterification of an unsymmetrical bis-dihydroxyketone such as 75 in an effort to selectively produce either 105 or 106 would be impractical and wasteful, because it would lead to a mixture of the two products (Scheme 35). The separation of these may be difficult, and in any case, it would probably require extensive chromatographic operations, rendering the synthetic route prohibitively expensive.

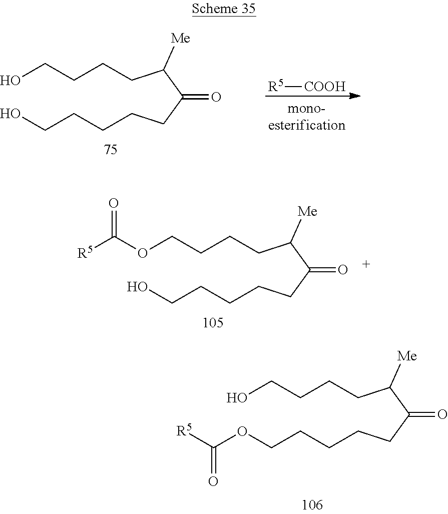

Conversely, a compound such as 14 of Scheme 6 lends itself nicely to highly selective mono-esterification, leading a single product 107 (Scheme 36). Release of the silyl group and esterification of the free OH in 108 results in formation of 109, which may be further reduced to 110. Ketone 109 and alcohol 110 can then be transformed into ionizable lipids by the methods shown in Schemes 30-33 above.

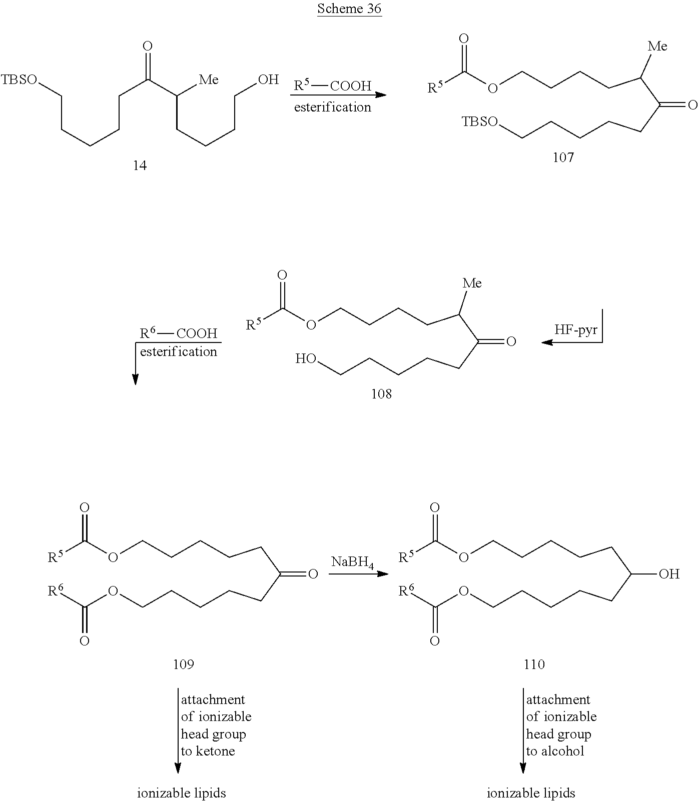

Ketodiacids of Formula XI or XII can be transformed into ionizable lipids starting with the esterification of the COOH groups with a lipophilic alcohol of Formula XIV. This is exemplified in Scheme 37 by the conversion of 37 and 83 into 111-112 by reaction with R⁶—OH in the presence of a condensing agent. The ketone group in 111-112 can be selectively reduced, e.g., by the use

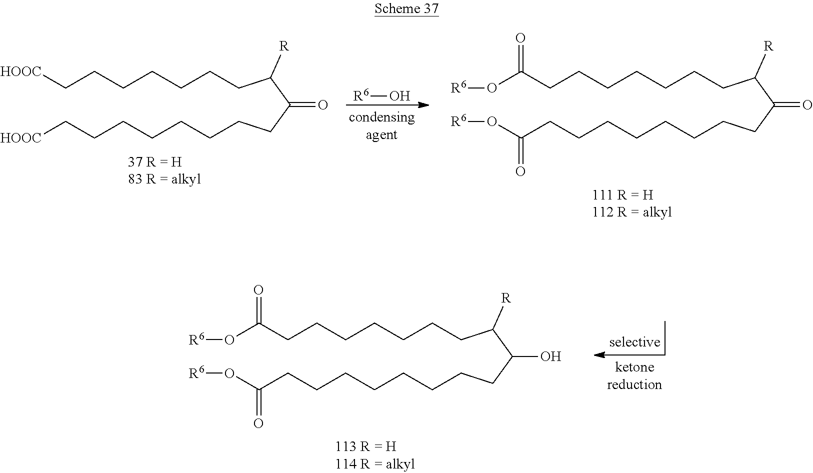

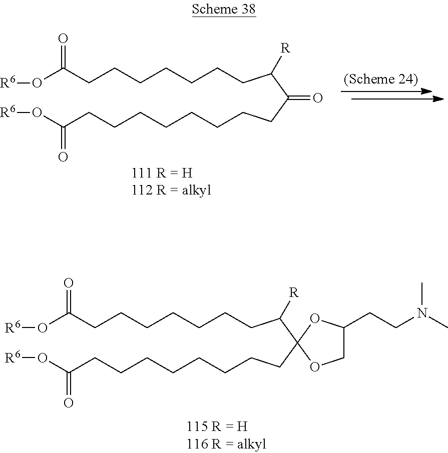

of NaBH₄ in an appropriate solvent, to produce alcohols 113-114. Compounds 111-112 and 113-114 can then be converted into ionizable lipids by the methods shown in Schemes 30-33 above. For example, ketones 111-112 may be transformed into ionizable lipids 115-116 by the method outlined earlier in Scheme 30 (Scheme 38). Ketones 111-112 can also be converted into ionizable lipids 117-118 (Scheme 39) via a reductive amination, as shown above in Schemes 31-32:

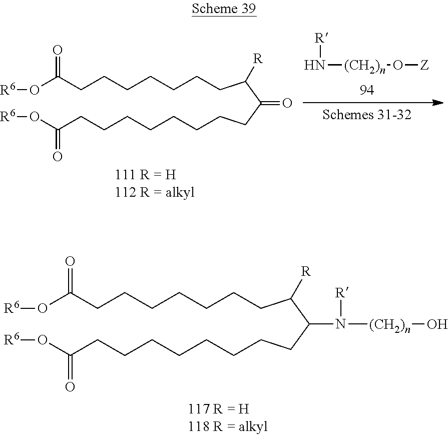

Alcohols 109-110 can be transformed into representative lipids 115-116 (Scheme 40) by the method shown earlier in Scheme 33:

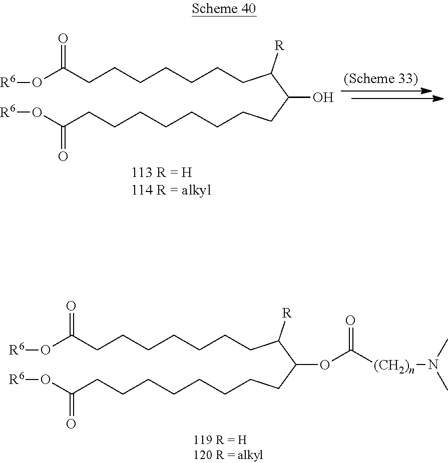

The foregoing examples are meant to be purely illustrative and should not be construed as limiting. Thus, the ketones and the alcohols produced through the methods in this disclosure may be used to prepare a variety of other ionizable lipids not shown in the above examples using methods, processes, and techniques that are well known to those of skill in the art.

### Example 7: Representative Claisen Condensation of Lactones

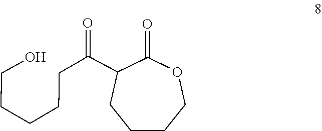 [SMILES: O=C(CCCCCO)C1CCCCOC1=O]

3-(6-hydroxyhexanoyl)oxepan-2-one, 8. To a solution of caprolactone (19.4 mL, 175.2 mmol, 1.0 equiv) and triethylamine (36.6 mL, 262.8 mmol, 1.5 equiv) in CH₂Cl₂ (150 mL) at −78° C. was added TiCl₄ (25 mL, 148.3079 mmol, 1.3 equiv) dropwise over 30 minutes via syringe pump. The resulting mixture was warmed up to room temperature and stirred for 5 h, then it was poured into ice cold phosphate buffer (pH 6.8). The organic layer was removed, and the aqueous layer was further extracted with CH₂Cl₂ (6×50 mL). The combined extracts were washed with brine, dried (Na₂SO₄), filtered, and concentrated in vacuo. The residue was purified by silica gel flash column chromatography with 9:1 CH₂Cl₂: acetone to afford 8 (18.34 g, 80.3 mmol, 92%) as a colorless oil. ¹H NMR (300 MHz, CDCl₃) δ 4.39-4.13 (m, 2H), 3.67-3.55 (m, 3H), 2.61 (dt, J=17.4, 7.4, 1H), 2.44 (dt, J=17.4, 7.2, 1H), 2.17-1.85 (m, 3H), 1.85-1.65 (m, 2H), 1.65-1.46 (m, 6H), 1.36 (qd, J=7.7, 7.3, 3.8, 2H).

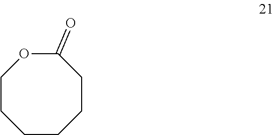

Oxocan-2-one, 21. To a solution of meta-chloroperoxybenzoic acid (16.2 g, 94.1 mmol, 2 equiv). After stirring for 5 days at 40° C., the reaction mixture was filtered, sequentially washed with saturated aq. Na₂S₂O₃, saturated aq. NaHCO₃ and water, dried (Na₂SO₄) and concentrated in vacuo to afford 21 (6.0 g, 47.0 mmol, ˜100%) as a colorless oil that was used for the next step without purification. ¹H NMR (300 MHZ, CDCl₃) δ 4.32 (t, J=5.6, 2H), 2.52 (t, J=6.4, 2H), 1.81 (m, 4H), 1.57 (m, 4H).

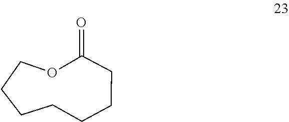

Oxonan-2-one, 23. To a solution of meta-chloroperoxybenzoic acid (2.8 g, 15.9 mmol, 2 equiv) in CH₂Cl₂ (10 mL) at 0° C. was added cyclooctanone (1.0 g, 7.9 mmol, 1.0 equiv). After stirring for 5 days at 40° C., the reaction mixture was filtered, sequentially washed with saturated aq. Na₂S₂O₃, saturated aq. NaHCO₃ and water, dried (Na₂SO₄) and concentrated in vacuo to afford 23 (1.1 g, 7.7 mmol, ˜97%) as a colorless oil and used for the next step without further purification. ¹H NMR (300 MHZ, CDCl₃) δ 4.27 (t, J=5.7, 2H), 2.27 (t, J=6.3, 2H), 1.39-1.73 (m, 10H). The NMR data was constituted with literature report.

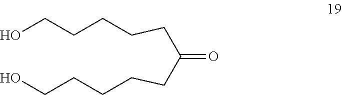 [SMILES: O=C(CCCCCO)CCCCCO]

1,11-dihydroxyundecan-6-one, 19. To a solution of caprolactone (19.4 mL, 175.2 mmol, 1.0 equiv) and triethylamine (44 mL, 315.4 mmol, 1.5 equiv) in CH₂Cl₂ (100 mL) at −78° C. was added TiCl₄ (28.8 mL, 262.8 mmol, 1.3 equiv) dropwise over 30 minutes via syringe pump. The resulting mixture was allowed to warm to room temperature and stirred for 5 h, then it was poured into cold water (100 mL). The organic layer was removed and the aqueous layer was further extracted with 95:5 CH₂Cl₂:MeOH (5×100 mL). The combined extracts were evaporated in vacuo. The residue was taken up into 1 M HCl (50 mL) and heated at 60° C. for 5 h, then the solution was extracted with 95:5 CH₂Cl₂:MeOH (95:5) (5×100 mL). The combined organic layers were washed with brine, dried (Na₂SO₄), filtered, and concentrated in vacuo. The solid residue was purified by crystallization using ethyl ether: hexanes (2:1) to afford 19 (16.1 g, 79.6 mmol, 91%) as an off white solid, m.p. 57° C. (lit.⁷⁷ m.p. 58.5° C.). ¹H NMR (300 MHz, CDCl₃) δ 3.62 (t, J=6.5, 4H), 2.39 (t, J=7.2, 4H), 1.57 (m, 8H), 1.33 (m, 4H).

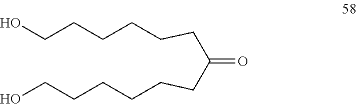 [SMILES: O=C(CCCCCCO)CCCCCCO]

1,13-dihydroxytridecan-7-one, 58. To a solution of lactone 21 (300 mg, 2.34 mmol, 1.0 equiv) and triethylamine (0.49 mL, 3.51 mmol, 1.5 equiv) in CH₂Cl₂ (15 mL) at −78° C. was added TiCl₄ (0.33 mL, 3.04 mmol, 1.3 equiv) dropwise over 30 minutes via syringe pump. The resulting mixture was allowed to warm to room temperature and stirred for 2 h, then it was poured into cold water (50 mL). The organic layer was separated, and the aqueous layer was further extracted with CH₂Cl₂ (3×25 mL). The combined extracts were evaporated in vacuo and to the residue was taken up into 1 M HCl (50 mL). The resulting mixture was heated at 60° C. on the rotary evaporator for 2 h, then extracted with 95:5 CH₂Cl₂:MeOH (5×100 mL). The combined extracts were washed with brine, dried (Na₂SO₄), filtered, and concentrated in vacuo. The residue was purified by crystallization with diethyl ether: hexanes (1:1) to afford 58 (180 mg, 0.78 mmol, 67%) as an off-white solid, m.p. 62-65° C.; (300 MHz, CDCl₃) δ 3.61 (t, J=6.5, 4H), 2.37 (t, J=7.3, 4H), 1.88 (s, 2H), 1.54 (m, 8H), 1.32 (m, 8H).

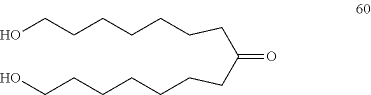 [SMILES: O=C(CCCCCCCO)CCCCCCCO]

1,15-dihydroxypentadecan-8-one, 60. To a solution of lactone 23 (2.1 g, 7.0 mmol, 1.0 equiv) and triethylamine (1.5 mL, 10.6 mmol, 1.5 equiv) in CH₂Cl₂ (25 mL) at −78° C. was added TiCl₄ (1.0 mL, 9.14 mmol, 1.3 equiv) dropwise over 30 minutes via syringe pump. The resulting mixture was stirred at room temperature for 5 h, then it was poured into cold water. The organic layer was removed and the aqueous layer was further extracted with 95:5 CH₂Cl₂:MeOH (5×100 mL). The combined extracts were evaporated in vacuo and the residue was taken up into 1 M HCl (50 mL). The mixture was heated at 60° C. on the rotary evaporator for 5 h, then extracted with 95:5 CH₂Cl₂:MeOH (5×100 mL). The combined extracts were washed with brine, dried (Na₂SO₄), filtered, and concentrated in vacuo. The residue was purified by crystallization using ethyl ether: hexanes (1:1) to afford 60 (1.2 g, 4 mmol, 68%) as an off-white solid. (300 MHZ, CDCl₃) δ 4.29 (s, 2H), 3.62 (t, J=6.6, 4H), 2.33 (t, J=7.5, 4H), 1.57 (dq, J=20.4, 6.9, 8H), 1.34 (q, J=4.0, 12H).

### Example 8: Representative Alkylation Reactions of the Products of Claisen Condensation of Lactones

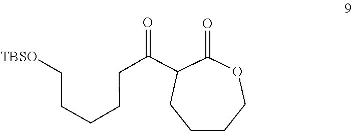 [SMILES: CC(C)(C)[Si](C)(C)OCCCCCC(=O)C1CCCCOC1=O]

3-(6-((tert-butyldimethylsilyl)oxy) hexanoyl) oxepan-2-one, 9. A solution of tert-butyl(chloro)-dimethylsilane (363 mg, 2.41 mmol, 1.1 equiv) in CH₂Cl₂ (4 mL) was added dropwise to a solution of compound 8 (500 mg, 2.19 mmol, 1.0 equiv) and imidazole (327 mg, 4.82 mmol, 2.2 equiv) in CH₂Cl₂ (25 mL) at room temperature. The mixture was stirred overnight at room temperature, then it was quenched with water (25 mL). The organic layer was removed and the aqueous layer was further extracted with CH₂Cl₂ (3×25 mL). The combined extracts were washed with brine (sat. solution), dried (Na₂SO₄), filtered, and concentrated in vacuo to afford 9 (750 mg, 2.19 mmol, 100%) as a colorless oil that was used for the next step without purification. ¹H NMR (300 MHz, CDCl₃) δ 4.36-4.16 (m, 2H), 3.64-3.54 (m, 3H), 2.60 (dt, J=17.4, 7.5, 1H), 2.43 (dt, J=17.3, 7.4, 1H), 2.12-1.22 (m, 12H), 0.85 (s, 9H), 0.01 (s, 6H).

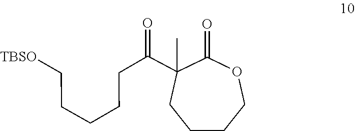 [SMILES: CC1(C(=O)CCCCCO[Si](C)(C)C(C)(C)C)CCCCOC1=O]

3-(6-((tert-butyldimethylsilyl)oxy) hexanoyl)-3-methyloxepan-2-one, 10. To a 3.4 mmol, 1.0 equiv) in N,N-dimethylformamide (3 mL) at room temperature was added a solution of compound 9 (1.2 g, 3.4 mmol, 1 equiv) in N,N-dimethylformamide (1 mL). The resulting mixture was stirred at room temperature for 30 min, then MeI (0.19 mL, 3.1 mmol, 0.9 equiv) was added. The reaction mixture was stirred at the room temperature for 5 hr, then it was cooled to 0° C., quenched with water (10 mL) and extracted with diethyl ether (3×25 mL). The combined extracts were washed with brine, dried (Na₂SO₄), filtered, and concentrated in vacuo. The residue was purified by flash column chromatography on silica gel with ethyl acetate: hexanes (1:3) to afford 10 (1.02 g, 2.86 mmol, 83%) as a colorless oil. ¹H NMR (300 MHz, CDCl₃) δ 4.22 (dt, J=13.0, 5.0, 2.2, 1H), 3.82 (ddd, J=13.0, 10.3, 1.3, 1H), 3.57 (t, J=6.4, 2H), 2.59 (dt, J=17.8, 7.2, 1H), 2.39 (dt, J=17.8, 7.2, 1H,), 2.19 (dq, J=12.2, 3.8, 3, 1H), 1.86-1.45 (m, 11H), 1.40 (s, 3H), 0.86 (s, 9H), 0.01 (s, 6H).

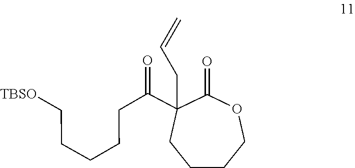 [SMILES: C=CCC1(C(=O)CCCCCO[Si](C)(C)C(C)(C)C)CCCCOC1=O]

3-allyl-3-(6-((tert-butyldimethylsilyl)oxy) hexanoyl) oxepan-2-one, 11. To a stirred suspension of sodium hydride (17 mg, 0.70 mmol, 1.2 equiv) in THF at 0° C., a solution of lactone 9 (200 g, 0.583 mmol, 1.0 equiv) in THF was added dropwise. The resulting mixture was stirred at the same temperature for 30 min, followed by slow addition of allyl bromide (75 μL, 0.875 mmol, 1.5 equiv). The reaction mixture was allowed to warm up to room temperature and stirred for 18 hr. The reaction mixture was quenched with water (25 mL) and extracted with diethyl ether (3×25 mL). The combined organic layers were washed with brine (sat. solution), dried (Na₂SO₄), filtered, and concentrated in vacuo. The resulting residue was purified by flash column chromatography with 1:3 ethyl acetate: hexanes to afford 11 (138 mg, 0.361 mmol, 62%) as a colorless oil. ¹H NMR (300 MHz, CDCl₃) δ 5.68 (ddt, J=17.6, 10.6, 7.2, 1H), 5.06 (dd, J=17.6, 10.6, 2H), 4.20 (dt, J=13.1, 3.9, 1H), 3.83 (ddd, J=12.4, 9.9, 2, 1H), 3.57 (t, J=6.2, 2H), 2.65-2.43 (m, 4H), 2.23-2.15 (m, 1H), 1.87-1.44 (m, 9H), 1.35-1.23 (m, 2H), 0.86 (s, 9H), 0.01 (s, 6H).

### Example 9: Representative Desilylation Reaction with Retention of the Lactone Ring

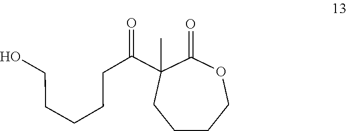 [SMILES: CC1(C(=O)CCCCCO)CCCCOC1=O]

3-(6-hydroxyhexanoyl)-3-methyloxepan-2-one, 13. Pyridine-HF complex (70% w/w, 183 μL, 4 equiv) and pyridine (200 μL) were added to a cold (0° C.) solution of lactone 10 (562 mg, 1.6 mmol, 1 equiv) in 1 mL of anhydrous THF. The mixture was allowed to warm to ambient temperature and stirred for 1 h, then it was poured into ice cold phosphate buffer (pH 6.8) and extracted with CH₂Cl₂ (6× 4 mL). The combined extracts were dried (Na₂SO₄), filtered, and concentrated in vacuo. The residue was purified by silica gel flash column chromatography with 9:1 CH₂Cl₂: acetone to afford 13 (300 mg, 1.24 mmol, 78%) as a colorless oil. ¹H NMR (300 MHz, CDCl₃) δ 4.20 (m, J=13.1, 3.9, 1H), 3.83 (m, 1H), 3.57 (t, J=6.2, 2H), 2.44 (m, 2H), 2.00-1.23 (cm, ˜12H), 1.41 (s, 3H).

### Example 10: Representative Lactone Hydrolysis with Retention of the Silyl Group

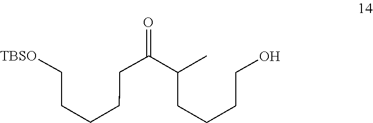 [SMILES: CC(CCCCO)C(=O)CCCCCO[Si](C)(C)C(C)(C)C]

11-((tert-butyldimethylsilyl)oxy)-1-hydroxy-5-methylundecan-6-one, 14 To a stirred solution of lactone (240 mg, 0.67 mmol, 1.0 equiv) in THF (3 mL) and water (3 mL) at room temperature, lithium hydroxide (65 mg, 2.68 mmol, 4 equiv) was added. The resulting mixture was stirred at the same temperature for 18 hr. Then an aqueous solution of HCl (1 M, 3 mL) was added and stirred at room temperature for 8 hr. Then water (10 mL) was added and extracted with dichloromethane (3×25 mL). The combined organic layers were washed with brine (sat. solution), dried (Na₂SO₄), filtered, and concentrated in vacuo. The resulting residue was purified by flash column chromatography eluting with ethyl acetate: hexanes (1:3) to afford the title compound (154 mg, 0.47 mmol, 70%) as a colorless oil. (300 MHz, CDCl₃) δ 3.61 (t, 6.5, 4H), 3.57 (t J=6.5, 2H), 2.49 (m, 1H), 2.41 (m, 2H), 1.60-1.45 (m, 8H), 1.34-1.22 (m, 4H), 1.04 (d, J=7.0, 3H), 0.86 (s, 9H), 0.02 (s, 6H).

### Example 11: Representative Lactone Hydrolysis with Simultaneous Desilylation

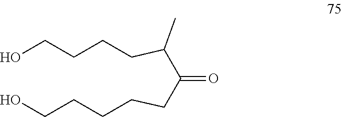

1,11-dihydroxy-5-methylundecan-6-one, 75 To a stirred solution of lactone 10 (82 mg, 0.31 mmol, 1.0 equiv) in THF (2 mL) at room temperature, lithium hydroxide (30 mg, 1.26 mmol, 4 equiv) was added. The resulting mixture was stirred at the same temperature for 18 hr. Then an aqueous solution of HCl (1 M, 3 mL) was added and stirred at room temperature for 5 hr. Then water (10 mL) was added and extracted with dichloromethane (3×25 mL). The combined organic layers were washed with brine (sat. solution), dried (Na₂SO₄), filtered, and concentrated in vacuo. The resulting residue was purified by flash column chromatography eluting with ethyl acetate: hexanes (1:3) to afford 75 (50 mg, 0.22 mmol, 75%) as a colorless oil. ¹H NMR (300 MHz, CDCl₃) δ 3.61 (t, J=6.5, 4H), 2.50 (t, J=6.7, 2H), 2.41 (m, 1H), 1.52-1.25 (m, 12H), 1.04 (d, J=7.0, 3H).

### Example 12: Representative Sulfonylation Reactions of the Products of Claisen Condensation of Lactones

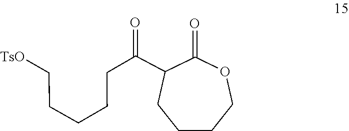 [SMILES: Cc1ccc(S(=O)(=O)OCCCCCC(=O)C2CCCCOC2=O)cc1]

6-oxo-6-(2-oxooxepan-3-yl)hexyl 4-methylbenzenesulfonate, 15. To a solution of lactone 8 (1.0 g, 4.4 mmol, 1.0 equiv), pyridine (0.5 mL, 5.7 mmol, 1.3 equiv) and N,N-dimethyl aminopyridine (cat. amount) in CH₂Cl₂ (10 mL) at room temperature was added p-toluenesulfonyl chloride (1.3 g, 6.6 mmol, 1.5 equiv). The resulting mixture was stirred at room temperature for 5 h, then it was quenched with water (25 mL). The organic layer was removed, and the aqueous layer was further extracted with CH₂Cl₂ (3× 25 mL). The combined organic layers were washed with brine, dried (Na₂SO₄), filtered, and concentrated in vacuo. The residue was purified by flash column chromatography eluting with 99:1 CH₂Cl₂: acetone to afford 15 (1.4 mg, 3.7 mmol, 84%) as a colorless oil. ¹H NMR (300 MHZ, CDCl₃) δ 7.75 (d, J=8.3, 2H), 7.32 (d, J=8.1, 2H), 4.33 (dd, J=12.5, 4.1, 1H), 4.20 (dd, J=12.5, 10, 1H), 3.98 (t, J=6.4, 3H), 3.60 (dd, J=11, 2, 1H), 2.57 (dd, J=17.4, 7.3, 1H), 2.42 (s, 3H), 2.36 (dd, J=17.3, 7.4, 1H), 2.13-1.91 (m, 2H), 1.80-1.151 (m, 10H), 1.36-1.23 (m, 2H).

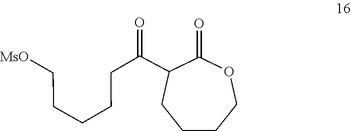 [SMILES: CS(=O)(=O)OCCCCCC(=O)C1CCCCOC1=O]

6-oxo-6-(2-oxooxepan-3-yl)hexyl methanesulfonate, 16. To a solution of lactone 8 (10 g, 43.8 mmol, 1.0 equiv), pyridine (4.6 mL, 57.0 mmol, 1.3 equiv) in CH₂Cl₂ (20 mL) at room temperature was added methanesulfonyl chloride (4.41 mL, 57.0 mmol, 1.3 equiv). The resulting mixture was stirred at room temperature for 5 hrs then it was quenched with water (25 mL). The organic layer was removed, and the aqueous layer was extracted with CH₂Cl₂ (3× 25 mL). The combined extracts were washed with brine, dried (Na₂SO₄), filtered, and concentrated in vacuo to afford 16 (13.4 gm, 43.8 mmol, ca. 100%) as a colorless oil and used for the next step without purification. (300 MHZ, CDCl₃) δ 4.33 (dd, J=12.5, 4.1, 1H), 4.26 (dd, J=12.5, 10, 1H), 4.23 (t, J=6.4, 2H), 3.66 (dd, J=11, 2, 1H), 3.01 (s, 3H), 2.66 (dd, J=17.4, 7.3, 1H), 2.47 (dd, J=17.3, 7.4, 1H), 2.18-1.91 (m, 2H), 1.85-1.38 (m, 10H).

### Example 13: Representative Sulfonate Displacement

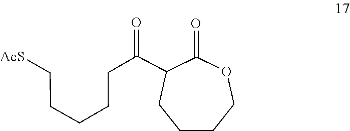

S-(6-oxo-6-(2-oxooxepan-3-yl) hexyl) ethanethioate, 17. To a solution of crude tosylate 15 (1.40 g, nominally 3.67 mmol) and TEA (1.3 mL, 964 mg, 9.5 mmol, 2.6 equiv) in DMF (10.0 mL) was added thioacetic acid (668 uL, 722 mg, 9.5 mmol, 2.6 equiv). The mixture was stirred at 60° C. for 16 hours, diluted with water (50.0 mL) and extracted with hexanes (3×40.0 mL). The combined extracts were washed (brine), dried (Na₂SO₄) and concentrated to yield 17 (745 mg, 71% from compound 8 over 2 steps). ¹H NMR (400 MHZ, CDCl₃) δ 4.33 (dd, J 12.5, 4.1, 1H), 4.20 (dd, J 12.5, 10, 1H), 3.60 (dd, J 11, 2, 1H), 2.84 (t, J=7.3 Hz, 2H), 2.56 (dd, J 17.4, 7.3, 1H), 2.42 (s, 3H), 2.36 (1H, dd, J 17.3, 7.4, CH₂CO), 2.31 (s, 3H), 2.13-1.91 (2H, m, CH₂), 1.80-1.151 (10H, m, 4×CH₂), 1.36-1.23 (2H, m, CH₂).

### Example 14: Representative Conversion of Dicarboxylic Acid Diesters into Dicarboxylic Acid Monoesters

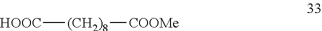

10-methoxy-10-oxodecanoic acid (monomethyl sebacate), 33. A solution of Ba(OH)₂ (10.5 g, 0.0614 mol) and commercial dimethyl sebacate (23.5 g, 0.102 mol) in MeOH (100 mL) was stirred for 2 hours at room temperature, whereupon a thick white precipitate separated. The solid was collected by filtration and washed with MeOH, then it was suspended in 4 N HCl and extracted with DCM (3×30.0 mL). The combined extracts were washed (brine), dried (Na₂SO₄), and concentrated to yield monomethyl sebacate (14.1 g, 64%). ¹H NMR (400 MHZ, CDCl₃) δ 3.66 (s, 3H), 2.34 (t, J=7.5 Hz, 2H), 2.30 (t, J=7.5 Hz, 2H), 1.75-1.52 (m, 4H), 1.43-1.25 (m, 8H).

### Example 15: Representative Claisen Condensation of Dicarboxylic Acid Monoesters

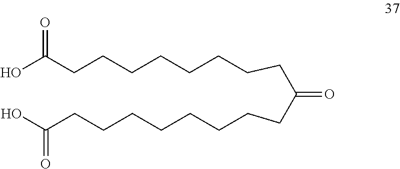 [SMILES: O=C(O)CCCCCCCCC(=O)CCCCCCCCC(=O)O]

10-oxononadecanedioic acid, 37. A solution of TiCl₄ (4.42 mL, 40.2 mmol) in DCM (10.0 mL) was added dropwise over the course of 10 minutes to a cold (0° C.) solution of monomethyl sebacate (2.5 g, 11.5 mmol) and triethylamine (8.01 mL, 57.5 mmol) in DCM (30.0 mL), under argon. Upon completion of the addition, the mixture was warmed to rt and stirred for 90 minutes. Water (15.0 mL) was added and the mixture was extracted with n-butanol (3×35.0 mL). The combined butanol portions were washed (brine), dried (Na₂SO₄) and concentrated. The residue was dissolved in EtOH (30.0 mL) and 4 N NaOH (8.00 mL) was added. The mixture was stirred at rt for 2 hours, cooled to room temperature, washed with Et₂O (2×30.0 mL) and acidified to pH 1 with conc. HCl. The mixture was concentrated to roughly 40% volume and extracted with 3:1 DCM: IPA (3×35.0 mL). The combined organics were washed (brine), dried (Na₂SO₄) and concentrated. The residue was purified by silica chromatography (0->20% MeOH in DCM) to yield 37 (707 mg, 36%). ¹H NMR (400 MHZ, CDCl₃) δ 2.44-2.38 (m, 4H), 2.37-2.30 (m, 4H), 1.69-1.55 (m, 24H). MS (negative ion ESI): m/z 341 [M−1]⁻.

### Example 16: Representative Synthesis of Ketodiacids Via Ketene Dimers

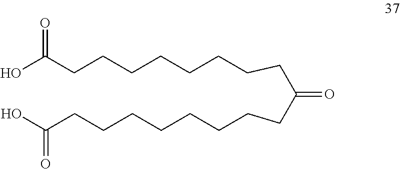 [SMILES: O=C(O)CCCCCCCCC(=O)CCCCCCCCC(=O)O]

10-oxononadecanedioic acid, 37. A solution of monomethyl adipate, 33, (6.5 g, 30 mmol) in SOCl₂ (5 mL) was heated to reflux for 2 minutes then cooled to room temperature. Excess SOCl₂ was removed under vacuum. The residue was dissolved in toluene (5 mL) and concentrated to remove any remaining SOCl₂, yielding the crude acid chloride 39 (7 g, quantitative), which was used in the next step without purification. ¹H NMR (400 MHZ, CDCl₃) δ 3.66 (s, 3H), 2.92 (t, J=7.0 Hz, 2H), 2.33 (t, J=7.1 Hz, 2H), 1.87-1.60 (m, 12H). Neat TEA (4.2 mL, 30 mmol) was added dropwise over the course of 3 minutes to a stirring solution of the above acid chloride (7 g, 30 mmol) in toluene (50.0 mL) at 0° C. under an atmosphere of nitrogen. The reaction was warmed to 35° C. and stirred for 15 minutes, then cooled to room temperature and stirred for an additional 30 minutes, at which point a thick white precipitate had formed. The mixture was filtered through a pad of Celite,® and the solid precipitate was washed with more toluene (15.0 mL). The combined filtrates were concentrated to yield methyl 9-(3-(8-methoxy-8-oxooctyl)-4-oxooxetan-2-ylidene) nonanoate, 40, as a mixture of E- and Z-isomers. This material was used directly in the next step without further purification. ¹H NMR (400 MHZ, CDCl₃) δ 4.75 (dt, 1H, J₁=7.7, J₂=1.3 Hz), 3.67 (s, 6H, double peak), 3.99 (br t, 1H, J=6.9 Hz), 2.40-2.29 (m, 4H), 2.19 (br q, 2H, J=7.5 Hz), 1.89-1.55 (m, 22H). The compound was suspended in 2 N aq. KOH (25.0 mL) and heated at reflux for 6 hours, whereupon the solution became homogenous. The cooled solution was washed with Et₂O (2×15.0 mL), and the ether extracts were discarded. The solution was then acidified with conc. HCl to pH 2. The aqueous layer was then kept at 0° C. for 1 hour, during which time a precipitate formed. The solid was collected by suction filtration to yield 10-oxononadecanedioic acid, 37, as an off white solid (3.4 g, 67% over two steps). ¹H NMR (400 MHZ, CDCl₃) δ 2.44-2.38 (m, 4H), 2.37-2.30 (m, 4H), 1.7-1.5 (m, 24H). MS (negative ion ESI): m/z 341 [M−1]⁻.

### Example 17: Representative Selective Reduction of Dicarboxylic Acid Monoesters to Terminal Hydroxyesters

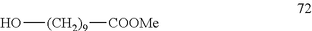

methyl 10-hydroxydecanoate, 72. Commercial BH₃□SMe₂ (7.3 mL, 77.1 mmol) was added via syringe to a cold (0° C.) solution of monomethyl sebacate (13.4 g, 61.8 mmol) in THF (100 mL), under nitrogen atmosphere. The mixture was warmed to room temperature and stirred for 90 minutes, at which point ¹H NMR confirmed consumption of starting material. The mixture was cooled to 0° C. and carefully quenched with H₂O (20.0 mL). The mixture was then extracted with CH₂Cl₂ (3×40.0 mL). The combined extracts were washed with brine, dried (Na₂SO₄), and concentrated to yield 72 (11.7 g, 94%). ¹H NMR (400 MHZ, CDCl₃) δ 3.64 (s, 3H), 3.60 (t, J=6.8 Hz, 2H), 2.27 (t, J=1.2 Hz, 2H), 1.62-1.47 (m, 4H), 1.28 (br s, 10H).

### Example 18: Representative OH Protection of Terminal Hydroxyesters

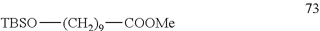

methyl 10-((tert-butyldimethylsilyl)oxy) decanoate, 73. A solution of 72 (229 mg, 1.06 mmol) TBDMS-Cl (176 mg, 1.17 mmol), and imidazole (86.6 mg, 1.27 mmol) in CH₂Cl₂ (3.00 mL) was stirred at room temperature, under nitrogen, for 18 hours. The 73 mixture then diluted with water (4.00 mL) and extracted with CH₂C12 (3×4.00 mL). The combined extracts were washed (brine), dried (Na₂SO₄), and concentrated. The residue was purified by silica chromatography (0-10% EtOAc in hexanes) to yield 73 (305 mg, 91%) as an oil. ¹H NMR (400 MHZ, CDCl₃) δ 3.66 (s, 3H), 3.59 (t, J=6.6 Hz, 2H), 2.30 (t, J=7.6 Hz, 2H), 1.69-1.59 (m, 2H), 1.55-1.44 (m, 2H), 1.36-1.25 (m, 8H), 0.89 (s, 9H), 0.04 (s, 6H).

### Example 19: Representative Claisen Condensation of Protected Derivatives of Terminal Hydroxyesters

1,19-dihydroxynonadecan-10-one, 67. A solution of TiCl₄ (0.16 mL, 1.45 mmol) in toluene (1.50 mL) was added dropwise to a cold (0° C.), stirred solution of 73 (306 mg, 0.97 mmol) and Bu₃N (0.413 mL, 1.74 mmol) in toluene (2.00 mL), under a nitrogen atmosphere. Upon

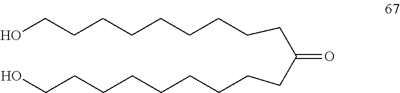 [SMILES: O=C(CCCCCCCCCO)CCCCCCCCCO]

completion of the addition, the mixture was warmed to rt and stirred for 90 minutes, then the reaction was quenched by the addition of water (3 mL). The mixture was extracted with CH₂Cl₂ (3×5.00 mL) and the combined extracts were washed (brine), dried (Na₂SO₄) and concentrated. The residue was dissolved in EtOH (4.00 mL) and 4 N NaOH (2.00 mL) was added. The mixture was stirred for 2 hours then acidified to pH 2 with conc. HCl. The mixture was extracted with CH₂Cl₂ (3×5.00 mL) and the combined extracts were washed (brine), dried (Na₂SO₄) and concentrated. The residue was purified by silica chromatography (0-15% MeOH in DCM) to yield 67 (247 mg, 85%). ¹H NMR (400 MHZ, CDCl₃): δ 3.62 (t, J=6.6 Hz, 4H), 2.37 (t, J=7.4 Hz, 4H), 1.62-1.50 (m, 10H), 1.32-1.23 (m, 18H).

### Example 20: Representative C-Alkylation of Product of Claisen Condensation

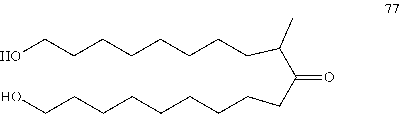 [SMILES: CC(CCCCCCCCO)C(=O)CCCCCCCCCO]

1,19-dihydroxy-9-methylnonadecan-10-one, 77. The Claisen condensation of 293 mg (0.93 mmol) of 73 was carried out as described in Example 14, except that the workup procedure was modified as follows. After stirring for 90 minutes after addition of the TiCl₄ solution, the reaction solution was slowly and carefully poured into vigorously stirred aqueous saturated NaHCO₃ solution (30 mL; foaming). The mixture was extracted with CH₂Cl₂ (3×10 mL) and the combined extracts were washed (brine), dried (Na₂SO₄) and concentrated to afford crude 74 (277 mg, 92%, oil, 3:1 mixture of keto- and enol tautomers). ¹H NMR (400 MHZ, CDCl₃, keto tautomer) δ 3.66 (s, 3H), 3.59 (t, J=6.6 Hz, 4H), 3.1 (t, J=6.2 Hz, 1H), 2.43 (t, J=7.6 Hz, 2H), 1.88 (m, 2H), 1.60-1.20 (cm, keto+enol resonances, ˜24H), 0.89 (br s, 18H), 0.04 (br s, 12H). Without purification, this material (assume 0.46 mmol) was dissolved in dry DMF (0.5 mL) and added to a dry DMF (0.5 mL) suspension of NaH (20 mg of 60% dispersion in mineral oil, ca. 12 mg, ca. 0.5 mmol), which had been previously been washed free of mineral oil with pentane, under argon. After stirring for 5 min, neat MeI (0.5 mmol) was injected and the mixture was stirred at room temperature for 2 hours. The reaction was quenched by careful addition of aqueous saturated NH₄Cl solution (0.5 ml) and extracted with a 1:1 mixture of diethyl ether and hexanes (3×5 mL). The combined extracts were washed with water (3×3 mL) and brine (1×3 mL), dried (Na₂SO₄) and concentrated to afford the crude methylated product, which was immediately dissolved in ethanol (4 mL) and treated with aqueous 10% NaOH solution (0.5 mL). The mixture was stirred at room temperature overnight, then it was acidified to pH 1 with concentrated aqueous HCl solution and evaporated to dryness. The temperature of the rotary evaporator bath was raised to 60° C. to induce decarboxylation. After 1 h, the residue in the flask was taken up with diethyl ether. The ether solution was filtered and evaporated to afford crude 77, which was purified by silica chromatography (0-15% MeOH in DCM) to yield 89 mg (59%) of product. ¹H NMR (400 MHZ, CDCl₃): δ 3.62 (t, J=6.6 Hz, 4H), 2.41 (t, J=7.4 Hz, 2H), 2.36 (m, 1H), 1.6-1.2 (m, 24H), 1.00 (d, J=6.9 Hz, 3H)

The foregoing examples are provided to exemplify the synthetic route of the disclosure and its advantages over known methodologies and are in no way meant to be limiting. As discussed previously, the method is broadly applicable to the production of building blocks for various ionizable lipid classes. Further, any document described herein, such as in the comparative examples, is not to be construed as an admission of prior art material to the patentability of the invention.

1. A method for producing one or more intermediates for the synthesis of one or more ionizable lipids, the method comprising:
(i) producing a beta-ketoacid by reacting a cyclic ester, a terminal hydroxyester or a derivative thereof, a dicarboxylic acid half ester, or an acid chloride derivative of the dicarboxylic acid half ester, in a condensation reaction, thereby producing the beta-ketoacid or a beta-ketoester that is hydrolyzed to produce the beta-ketoacid; and
(ii) decarboxylating the beta-ketoacid, thereby producing the one or more intermediates, wherein the one or more intermediates have a structure as defined by Formula A, 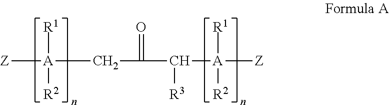
wherein:
each n is independently 2 to 12;
each A is, independently:
a carbon atom, in which case R¹ and R² are, independently, selected from H, alkyl, OH, O—Si(alkyl)₃, O-alkyl, S-alkyl, and N(alkyl)₂;
a sulfur or oxygen atom, in which case R¹ and R² are absent;
a C═C double bond of E or Z geometry, in which case R¹ and R² are, independently, H or
alkyl, O-alkyl, O—Si(alkyl)₃, S-alkyl,
R³ is H or a linear or branched alkyl group having from 1 to 20 carbon atoms, optionally incorporating (i) from 0 to 4 sulfur or oxygen atoms, (ii) from 0 to 3 C═C double bonds of E or Z geometry, (iii) OH, O-alkyl, O—Si(alkyl)₃, S-alkyl, N(alkyl)₂ substituents bonded to a carbon atom, and/or (iv) a linear or branched alkyl substituent having less than 6 carbon atoms; and
Z is independently OH or COOH.

2. The method of claim 1, wherein the beta-ketoester is alkylated prior to being hydrolyzed to produce the beta-ketoacid.

3. The method of claim 1, wherein the condensation reaction is a Claisen condensation of the cyclic ester, the terminal hydroxyester or the derivative thereof, or the dicarboxylic acid half ester to produce the beta-ketoacid or the beta-ketoester that is subsequently hydrolyzed to produce the beta-ketoacid.

4. The method of claim 3, wherein the Claisen condensation is carried out in the presence of a catalyst and a weak base.

5. The method of claim 4, wherein the catalyst is AlCl₃, GaCl₃, TiCl₄, ZrCl₄, HfCl₄ or SnCl₄.

6. The method of claim 5, wherein the catalyst is TiCl₄.

7. The method of any claim 4, 5 or 6, wherein the weak base is an amine.

8. The method of claim 7, wherein the amine is tributylamine or triethylamine.

9. The method of claim 1, wherein the condensation reaction comprises the conversion of the acid chloride to a ketene dimer by treatment with a weak base.

10. The method of claim 9, wherein the weak base is an amine.

11. The method of claim 10, wherein the amine is a trialkylamine.

12. The method of claim 11, wherein the trialkylamine is a tributylamine, triethylamine or diisopropylethylamine.

13. A method for producing one or more ionizable lipids, the method comprising:
(i) producing a beta-ketoacid by reacting a cyclic ester, a terminal hydroxyester or a derivative thereof, a dicarboxylic acid half ester, or an acid chloride derivative of the dicarboxylic acid half ester, in a condensation reaction, thereby producing the beta-ketoacid or a beta-ketoester that is hydrolyzed to produce the beta-ketoacid;
(ii) decarboxylating the beta-ketoacid, thereby producing one or more intermediates having a structure of Formula A; and
(iii) adding an ionizable head group moiety to (a) a ketone group of one or more intermediates having the structure of Formula A; or (b) a corresponding alcohol of the one or more intermediates, thereby producing the one or more ionizable lipids, wherein Formula A has a structure as defined below: 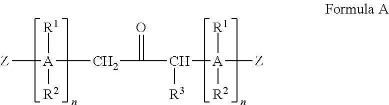
wherein:
each n is independently 2 to 12;
each A is, independently:
a carbon atom, in which case R¹ and R² are, independently, selected from H, alkyl, OH, O—Si(alkyl)₃, O-alkyl, S-alkyl, and N(alkyl)₂;
a sulfur or oxygen atom, in which case R¹ and R² are absent;
a C═C double bond of E or Z geometry, in which case R¹ and R² are, independently, H or
alkyl, O-alkyl, O—Si(alkyl)₃, S-alkyl,
R³ is H or a linear or branched alkyl group having from 1 to 20 carbon atoms, optionally incorporating (i) from 0 to 4 sulfur or oxygen atoms, (ii) from 0 to 3 C═C double bonds of E or Z geometry, (iii) OH, O-alkyl, O—Si(alkyl)₃, S-alkyl, N(alkyl)₂ substituents bonded to a carbon atom, and/or (iv) a linear or branched alkyl substituent having less than 6 carbon atoms; and
Z is independently OH or COOH.

14. The method of claim 13, wherein the beta-ketoester is alkylated prior to being hydrolyzed to produce the beta-ketoacid.

15. The method of claim 13, wherein the condensation reaction is a Claisen condensation of the cyclic ester, the terminal hydroxyester or the derivative thereof, or the dicarboxylic acid half ester to produce the beta-ketoacid or the beta-ketoester that is subsequently hydrolyzed to produce the beta-ketoacid.

16. The method of claim 15, wherein the Claisen condensation is carried out in the presence of a catalyst and a weak base.

17. The method of claim 16, wherein the catalyst is AlCl₃, GaCl₃, TiCl₄, ZrCl₄, HfCl₄ or SnCl₄.

18. The method of claim 17, wherein the catalyst is TiCl₄.

19. The method of any claim 16, 17 or 18, wherein the weak base is an amine.

20. The method of claim 19, wherein the amine is tributylamine or triethylamine.

21. The method of claim 13, wherein the condensation reaction comprises the conversion of the acid chloride to a ketene dimer by treatment with a weak base.

22. The method of claim 21, wherein the weak base is an amine.

23. The method of claim 22, wherein the amine is a trialkylamine.

24. The method of claim 23, wherein the trialkylamine is a tributylamine, triethylamine or diisopropylethylamine.
